SEQUENCE LISTING (6 sequences)

SEQ ID NO: 1 - DNA, 25 bp, Synthetic construct
  source 1..25: mol_type = other DNA
gacctgctgcatgggaattctgggg

SEQ ID NO: 2 - DNA, 19 bp, Synthetic construct
  source 1..19: mol_type = other DNA
ccagccagtttaccttaca

SEQ ID NO: 3 - DNA, 20 bp, Synthetic construct
  source 1..20: mol_type = other DNA
tgcttcatgcctttctacag

SEQ ID NO: 4 - DNA, 32 bp, Synthetic construct
  source 1..32: mol_type = other DNA
taatacgactcactatagggactgtagaaagg

SEQ ID NO: 5 - DNA, 20 bp, Synthetic construct
  source 1..20: mol_type = other DNA
aaaagcaccgactcggtgcc

SEQ ID NO: 6 - DNA, 193 bp, Synthetic construct
  source 1..193: mol_type = other DNA
gagcacacttcagaggcccccaggactcagcgctagcagagaaagccgacttaccacgcctctgaaggaaggagggataaggccactgtagaaagggatgaagcacgaacataccaaggcctgtgaaagcaaaggagagagaagttataggcgagagcacccttttaattttcctgatccttcataagctttc

(19) United States
(12) Patent Application Publication
(10) Pub. No.: US 2024/0218333 A1
(43) Pub. Date: Jul. 4, 2024

(54) GENERATING QUIESCENT HEPATIC STELLATE CELLS AND THEIR USE IN LIVER MODELS

(71) Applicant: The Cleveland Clinic Foundation, Cleveland, OH (US)

(72) Inventor: Xianfang WU, VIII, Cleveland, OH (US)

(21) Appl. No.: 18/528,348

(22) Filed: Dec. 4, 2023

Related U.S. Application Data
(60) Provisional application No. 63/385,873, filed on Dec. 2, 2022.

(51) Int. Cl. C12N 5/071

(52) U.S. Cl. CPC C12N 5/067; C12N 2500/25; C12N 2500/36; C12N 2500/38; C12N 2501/11; C12N 2501/113; C12N 2501/115; C12N 2501/155; C12N 2501/727; C12N 2506/45

(57) ABSTRACT

Provided herein are compositions, systems, kits, and methods for generating hPSC derived quiescent hepatic stellate cells (HSCs) and employing them in a liver model. In certain embodiments, methods and compositions are provided for maintaining the quiescent HSCs in a quiescent state for a time period.

The present application claims priority to U.S. Provisional application Ser. No. 63/385,873, filed Dec. 2, 2022, which is herein incorporated by reference in its entirety.

### SEQUENCE LISTING

The text of the computer readable sequence listing filed herewith, titled “CCF_41219_202_SequenceListing.xml”, created on Mar. 21, 2024, having a file size of 6,546 bytes, is hereby incorporated by reference in its entirety.

### FIELD

Provided herein are compositions, systems, kits, and methods for generating hPSC derived quiescent hepatic stellate cells (HSCs) and employing them in a liver model. In certain embodiments, methods and compositions are provided for maintaining the quiescent HSCs in a quiescent state for a time period.

### BACKGROUND

Nonalcoholic fatty liver disease (NAFLD), the most common form of chronic liver disease, encompasses a spectrum from steatosis to hepatocellular carcinoma (HCC) [1]. Disease progression is variable given the complex interplay between behavioral and genetic risk factors [1-3]. As such, the association of many single nucleotide polymorphisms (SNPs) with NAFLD arise in combination with metabolic insults. Strong hits include variants related to metabolic function like: lipid remodelling factor patatin-like phospholipase-domain containing protein 3 (PNPLA3) (rs738409 C>G) [4] and lipid droplet protein hydroxysteroid 17-beta dehydrogenase 13 (HSD17B13) (rs72613567 T>A) [5]. However, establishing mechanistic links between genetic variants and NAFLD requires human-relevant systems that recapitulate disease progression, are amenable to genetic manipulation and can respond to metabolic insults [6, 7].

The nonsynonymous rs738409 C>G SNP in PNPLA3 is strongly associated across the spectrum of NAFLD and accounts for the largest fraction of genetic predisposition to disease [4, 8]. PNPLA3 serves two roles in lipid metabolism as a triacylglycerol lipase in hepatocytes and a retinyl esterase in hepatic stellate cells (HSCs) [1, 9]. The isoleucine-to-methionine substitution at residue 148 (I148M) with rs738409 reduces enzymatic activity [10], suggesting that decreased lipid metabolism may promote NAFLD. However, Pnpla3-knockout mice do not develop hepatic steatosis whereas expressing the I148M mutant decreases hepatocyte triglyceride hydrolysis and increases de novo lipogenesis [11]. These conflicting results could be due to the partial identity or differential expression patterns of human and mouse PNPLA3 [11]. Thus, a human-relevant system is essential to understand how rs738409 contributes to NAFLD.

### SUMMARY

Provided herein are compositions, systems, kits, and methods for generating hPSC derived quiescent hepatic stellate cells (HSCs) and employing them in a liver model. In certain embodiments, methods and compositions are provided for maintaining the quiescent HSCs in a quiescent state for a time period.

In some embodiments, provided herein are methods of maintaining quiescence in quiescent hepatic stellate cells (HSCs) comprising: culturing quiescent HSCs in culture media for a time period such that the quiescent HSCs remain quiescent for the time period, wherein the time period is at least one day, and wherein the culture media comprises at least one of the following: fibroblast growth factor 1 (FGF1), fibroblast growth factor 2 (FGF2), and retinol.

In particular embodiments, the culture media comprises all three of FGF1, FGF2, and retinol. In other embodiments, the culture media further comprises epidermal growth factor (EGF) and/or lipids, wherein the EGF is optionally present in the culture media at a concentration of 5-120 ng/ml or 25-35 ng/ml, and optionally, wherein the lipids comprise a mixture of at least three of the following: arachidonic acid, linoleic acid, linolenic acid, myristic acid, oleic acid, palmitic acid stearic acid, cholesterol, Tween-80, tocopherol acetate and Pluronic F-68 (and optionally when said lipids make up about 0.5-4% of the culture media). In further embodiments, the time period is at least 2 days, or at least 7 days, or at least 14 days or at least 28 days, or at least 2 months. In further embodiments, the culture media comprises the FGF1, and wherein the FGF1 is present in the culture media at a concentration of about 1-80 ng/ml or 3-25 ng/ml. In other embodiments, the culture media comprises the FGF2 and wherein the FGF2 is present in the culture media at a concentration of about 2-40 ng/ml or 7-13 ng/ml. In additional embodiments, the culture media comprises the retinol and wherein the retinol is present in the culture media at a concentration of about 1-20 uM or about 3-7 uM.

In some embodiments, provided herein are composition, kits, and system comprising: a) quiescent hepatic stellate cells (HSCs) (e.g., derived from hPSCs, as described herein), and b) culture medium comprising at least one of the following: fibroblast growth factor 1 (FGF1), fibroblast growth factor 2 (FGF2), and retinol. In further embodiments, the culture media comprises all three of FGF1, FGF2, and retinol. In other embodiments, the culture media further comprises epidermal growth factor (EGF) and/or lipids, wherein the EGF is optionally present in the culture media at a concentration of 5-120 ng/ml or 25-35 ng/ml, and optionally, wherein the lipids comprise a mixture of at least three of the following: arachidonic acid, linoleic acid, linolenic acid, myristic acid, oleic acid, palmitic acid stearic acid, cholesterol, Tween-80, tocopherol acetate and Pluronic F-68 (and optionally when said lipids make up about 0.5-4% of the culture media). In further embodiments, the culture media comprises the FGF1, and wherein the FGF1 is present in the culture media at a concentration of about 1-80 ng/ml or 3-25 ng/ml. In particular embodiments, the culture media comprises the FGF2 and wherein the FGF2 is present in the culture media at a concentration of about 2-40 ng/ml or 7-13 ng/ml. In some embodiments, the culture media comprises the retinol and wherein the retinol is present in the culture media at a concentration of about 1-20 uM or about 3-7 uM.

In particular embodiments, provide herein are methods comprising: a) adding a single-cell suspension of human pluripotent stem cells (hPSCs) to a cell culture container containing a first cell-culture media, wherein the first cell-culture media optionally contains a p160ROCK inhibitor, wherein the p160ROCK inhibitor optionally comprises Y-27632; b) incubating at least a portion of the hPSCs such that at least about 10% confluence of the hPSCs is achieved; c) treating at least a portion of the hPSCs with: i) a second cell-culture media comprising at least one of the following: biotin, vitamin B12, and PABA, wherein the second cell-culture media optionally comprising RPMI 1640, ii) a first media supplement comprising at least 5, 6, 7, 8, 9, 10, 11, 12, 13, 14, 15, 16, 17, 18, 19, or 20 reagents from Table 1, and optionally wherein the first media supplement comprises B-27 media supplement without insulin, iii) optionally a second media supplement comprising L-alanyl-L-glutamine dipeptide, and wherein optionally the second media supplement comprises GlutaMax, iv) optionally a third media supplement comprising non-essential amino acids; v) optionally an inhibitor of the GSK-3 enzyme, wherein the inhibitor optionally comprises CHIR99021; d) treating at least a portion of the hPSCs with either: i) the second cell-culture media, the first media supplement, and BMP4, and/or ii) Mesoderm Induction Medium; and e) incubating at least a portion of the hPSCs such that mesoderm cells are generated.

In further embodiments, the methods further comprise: f) treating at least a portion of the mesoderm cells with: the second cell-culture media, the first media, BMP4, FGF1, insulin, transferrin, and selenite, and optionally ascorbic acid and/or dexamethasone; and g) culturing at least a portion of the mesoderm cells such that mesodermal progenitor cells are generated. In other embodiments, the methods further comprise: h) treating at least a portion of the mesodermal progenitor cells with: the second cell-culture media, the first media, insulin, transferrin, selenite, lipids, and FGF2, and optionally ascorbic acid, dexamethasone, and/or EGF, and i) culturing at least a portion of the mesodermal progenitor cells such that quiescent hepatic stellate cells (HSCs) are generated.

In particular embodiments, the lipids are selected from one or more of the following: arachidonic acid, linoleic acid, linolenic acid, myristic acid, oleic acid, palmitic acid, stearic acid, cholesterol, Tween-80, tocopherol acetate, Pluronic F-68 solubilized in cell culture water.

In some embodiments, provide herein are methods comprising: a) adding hPSC-derived hepatocytes (HEPs), hPSC-derived macrophages (Macs) and hPSC-derived hepatic stellate cells (HSCs) to a transwell container containing a first cell-culture media, wherein the Macs are added such that they are in a first compartment of the transwell container, and the HEPs and HSCs are added such that they are in a second compartment, wherein the first and second compartments are configured to allow secreted factors to diffuse between the first and second compartments; b) incubating the transwell container such that cell clusters are generated, wherein in each cell cluster comprises a plurality of HSCs surrounded by a larger plurality of HEPs, c) adding a second cell culture media to the first and second compartments of the transwell container, wherein the second culture media comprises basal maintenance media (BMM), and wherein optionally the BMM comprises Dulbecco's Modified Eagle Medium supplemented with knockout serum replacement (KOSR), B-27 supplement, heparin, transferrin, EGF, retinol, and dexamethasone; d) incubating the transwell container; e) supplementing the BMM with high levels of insulin, glucose, and free fatty acids which are optionally oleic acid and palmitic acid, to mimic lipotoxic liver conditions, optionally wherein the lipotoxic conditions are similar to plasma levels in NAFLD patients. In other embodiments, the methods further comprise: f) adding a candidate agent to the second transwell container, and incubating the transwell container. In additional embodiments, the methods further comprise: g) isolating the HSCs, the HEPs, and/or the Macs, and running as assay with such isolated cells to determine the impact of the candidate agent on such isolated cells.

In some embodiments, the lipids are selected from: at least one, two, three, or all of the following: non-animal derived fatty acids (e.g., 2 ug/ml, or 1-5 ug/ml, arachidonic and 10 ug/ml, or 5-20 ug/ml, each linoleic, linolenic, myristic, oleic, palmitic and stearic), about 0.22, or 0.1-0.8, mg/ml cholesterol, such as from New Zealand sheep's wool, about 2.2 mg/ml, or 1.0-5.0 mg/ml, Tween-80, about 70 ug/ml, or 30-100 ug/ml, tocopherol acetate and about 100 mg/ml, or 50-150 mg/ml, Pluronic F-68 solubilized in cell culture water). In certain embodiments, the lipids are selected from one or more of the following: non-animal derived fatty acids: arachidonic, linoleic, linolenic, myristic, oleic, palmitic and stearic acid, cholesterol, Tween-80, tocopherol acetate, Pluronic F-68 solubilized in cell culture water.

### DESCRIPTION OF THE FIGURES

FIG. 1. Generation of a hPSC-derived multicellular liver culture. (A) Top: scheme of HSCs differentiation protocol, followed by replating on plastic surface. Middle: representative images at specified stages. Bottom: staining of lipid droplets. (B) Auto-fluorescence (blue) of retinoic acid under UV light. Negative control: Mesoderm (MES) cells; Nuclei: Histone H3 (red). (C, D) Analysis of HSC markers by western blot (C) and RT-qPCR (D, n=4 for hPSC-derived HSCs and 2 technical replicates for each donor of pHSCs) in hPSC-derived HSCs from day 13 (qHSC) and 18 (aHSC) and primary HSCs (pHSCs) from two donors (pri-1 and pri-2) at early (p2) and late passage (p5). (E) Collagen secretions in supernatants from re-plated hPSC-HSCs. (F) Phagocytosis analysis of hPSC- and primary-macrophages using fluorescently pre-labeled E. coli (Green). Negative control: Cytochalasin D. (G) Cytochrome P450 activity in hPSC-derived hepatocytes (HEP, n=6) and primary human hepatocytes (PHH, n=4 technical replicates) from two donors. (H-I) Left: Scheme of multicellular liver culture (H). Right: staining of indicated cell-type markers. Staining of HEPs and HSCs in the bottom compartment (I). (J) Cell numbers at the indicated time post-coculture, n=3. Data are mean±SD and compared with Student's t-test (two-tailed) with Welch's corrections.

FIG. 2. Characterization of the multicellular liver culture. (A) Albumin secretion from three-cell (HEP/HSC/Mac), two-cell (HEP/HSC), and HEP monoculture, n=3. (B) Analysis of CYP3A4 and CYP2B6 transcripts in hepatocytes, n=4. (C) Analysis of LRAT transcript in HSCs from three-cell (HSC/HEP/Mac), two-cell (HSC/HEP, HSC/Mac) and HSC monoculture, n=4. (D) Staining of lipid droplets, hepatocytes (ALB), and HSCs (PCDH7). (E) Analysis of IL10 transcript in individual cell-type at three-weeks post-coculture, n=4. (F) Analysis of IL6 and TNF transcripts in macrophages, n=4. Positive control: IFNγ-treated macrophages. (G) Cytokines analysis in liver cultures treated with LPS (500 ng/ml) or control, with or without macrophages, n=4. Data are mean±S.D; Student's t-test (two-tailed) with Welch's corrections (B); One-way ANOVA with Tukey's multiple comparisons test (A, C, E-G).

FIG. 3. Modeling NAFLD in multicellular liver cultures. (A) Scheme of NAFLD liver cultures, with healthy (Heal) or lipotoxic milieu (Lipo). (B-E) Analysis of oil-red content (B, n=10), TAG (C, n=4), baseline glucose secretion (D, n=4) and insulin-induced glucose secretion (E, n=5) in hepatocytes. (F). Analysis of AKT, FOXO1, phosphorylated AKT and FOXO1 in insulin-treated hepatocytes. (G-H). Analysis of activation markers in HSCs (G) or cytokines in supernatants (H, n=4) from three-cell (HEP/HSC/Mac), two-cell (HSC/Mac, HSC/HEP) or HSC monocultures. (I). Analysis of TNF, TGFB, and IL6 transcripts in macrophages, n=4. (J). Analysis of CK18 fragments in supernatants with either glucose or fructose, n=3. (K). Scheme of liver cultures with hPSC-derived or primary cells (Pri). (L-O). Analysis of CHI3L1 and IL6 transcripts (macrophages, L, n=3), CXCL10 transcript and ATP levels (hepatocytes, M-N, n=3), and HSC markers (HSCs, O) in hPSC- or primary cells (two donors: Pri-1 and Pri-2)-derived liver cultures. Data are mean±S.D; Student's t-test (two-tailed) with Welch's corrections (B-D, I); One-way ANOVA with Tukey's multiple comparisons test (E, H, J, L-N).

FIG. 4. Generation of an isogenic pair of co-cultures harboring either PNPLA3WT or PNPLA3_{I148M}. (A). Introduction of rs738409 C>G into hPSC^{WT} to generate hPSC^{I148M} line. (B). Proliferation analysis of hPSC^{WT} and hPSC^{I148M} pluripotent cells, n=3. (C-F). Analysis of albumin secretion (hepatocytes, C, n=4); IL10 transcript (macrophages, n=3) and CXCL10 and IL6 transcripts (macrophages treated with LPS, E, n=3) and HSC markers (with or without LPS treatment, control: pHSCs from two donors at p3 without LPS treatment, F) in differentiated individual cell-types. (G). Expression of PNPLA3, PDGFRβ, ALB at three-weeks under healthy condition. (H-I). Analysis of IL10 transcript (macrophages, H, n=3) and LRAT (HSCs, I, n=3) in liver cultures under healthy condition. Week 0: macrophages and HSCs before co-culture. Data are mean±S.D; Student's t-test (two-tailed) with Welch's corrections.

FIG. 5. Enhanced susceptibility to NAFLD phenotypes in co-cultures harboring PNPLA3^{I148M}. (A). Analysis of oil-red intensity (left, n=10) and TAG (right, n=4) in purified hepatocytes. (B-D). Baseline glucose secretion (B, n=4), insulin-induced glucose secretion (C, n=4), and ATP level (D, n=3) in hepatocytes under lipotoxic condition. (E). Fibronectin (FN) secretion in culture supernatants, n=3. (F). Analysis of CHI3L1 transcript in macrophages, n=3. (G). Cytokines analysis in both liver cultures, n=3. Data are mean±S.D; One-way ANOVA with Tukey's multiple comparisons test.

FIG. 6. Elevated IL6/STAT3 activity in PNPLA3^{I148M} liver cultures and NAFLD patients. (A). Analysis of cytokine secretions at day 6 post-lipotoxic exposure, n=3. (B). Analysis of STAT3 and phosphorylated STAT3 in purified individual cells. (C). Heatmap: Top 30 DEGs across PNPLA3 wild-type, I148M heterozygous and homozygous groups. (D). GO analysis of DEGs between PNPLA3 I148M homozygous and wild-type patients (p<0.01). (E-F). Selected transcripts in pooled three-cell (E, n=4) or IL6-treated liver cultures (F, n=4). Data are mean±SD; Student's t-test (two-tailed) with Welch's corrections.

FIG. 7. IL6/STAT3 hyperactivation mediates the enhanced susceptibility to NAFLD phenotypes in PNPLA3^{I148M} liver cultures. (A-D). Analysis of TAG (A, n=4), ACC1 and FASN transcripts (B, n=3), baseline glucose secretion (C, n=4) in hepatocytes, and activation markers in HSCs (D) purified from liver cultures transduced with shRNA-control (sh-Ctrl) or shRNA-IL6 (sh-IL6). (E). Analysis of soluble IL6R (sIL6R) and gp130 (sgp130) in supernatants, n=3. (F-G). Analysis of ACC1 and FASN transcripts (F, n=3) and HSC activation markers (G) in liver culture treated with sgp130-Fc protein. (H). HSC activation markers in PNPLA3WT liver cultures supplemented with IL6. Data are mean±S.D; One-way ANOVA with Tukey's multiple comparisons test (A-C, E-F).

FIG. 8. IL6/STAT3 hyperactivation induced by NF-κB activation in PNPLA3^{I148M} liver cultures. (A). Analysis of IL6 transcript in purified individual cells, n=3. (B). Analysis of selected transcription factors in wild-type macrophages treated with both supernatants collected in FIG. 6A. (C). Analysis of IL6 transcript in macrophages collected from (B), n=4. (D). NF-kB luciferase reporter assay in transduced macrophages, n=4. Data are mean±S.D; Student's t-test (two-tailed) with Welch's corrections (C-D); One-way ANOVA with Tukey's multiple comparisons test (A).

FIG. 9. Generation of a hPSC-derived multicellular liver culture. (A). Heatmap of Z scores, given in transcripts per million (TPM), of stage-specific markers in cells at different stages of HSC differentiation from hPSCs and in hPSC-derived hepatocytes (HEP). (B). Transcript levels of the LPS receptor complex (CD14, TLR4, and LY96) in hPSC-derived quiescent HSCs, pooled two donors of primary HSCs, and hPSC-derived macrophages as determined by RT-qPCR. Shown are mean±SD from 4 independent experiments, after being normalized to values in hPSCs. (C). Transcript levels of the IL6 and CXCL10 in hPSC-derived quiescent HSCs, two donors of primary HSCs treated with LPS (500 ng/ml) or vehicle control for 12 hrs analyzed by RT-qPCR. Shown are mean±SD from 4 independent experiments for hPSC-derived HSCs, from 3 independent experiments for two donors of primary HSCs, after being normalized to values in corresponding vehicle control. (D). Western blot analysis of HSC activation markers COL1A1, FN, and ACTA2 in hPSC-derived HSCs treated with TGFβ1 (5 ng/ml), FBS (10% in DMEM), PDGF (30 ng/ml), or vehicle control for 72 hrs. RPS11 was used as a loading control. (E). Representative immunofluorescent images of COL1A1, ACTA2, HSP47, VIM, and PDGFRβ in cultured primary HSCs (passage 5) and FBS-activated hPSC-derived HSCs (passage 2) (scale bars, 100 μm). (F). hPSC-derived HSCs from day 13 (qHSC) and day 18 (aHSC) were compared to primary human HSCs (pHSCs) from two different donors (pri-1 and pri-2). Transcript levels of Desmin were analyzed by RT-qPCR Shown are mean±SD from 4 independent replicates for hPSC-derived HSCs cells and 2 technical replicates for each donor of pHSC. (G). hPSC-derived HSCs and primary HSCs were treated with PDGF (25 ng/ml), FBS (10%), or Y-27532 (20 μM) for 48 hrs. Closure distances were calculated as percentage with respect to time=0 distance. Shown are mean±SD from 3 independent experiments. (H). Flow cytometry analysis of surface expression of selected macrophage markers in hPSC-derived macrophages. Shown values are mean±SD from 3 independent experiments. (I). Transcript levels of CXCL10, IL6, IL1B in hPSC-derived and primary Kupffer cells/macrophages (mixture of three donors) to IFNγ (10 ng/ml), influenza A virus (FluA, 10⁵ particles/ml), LPS (100 ng/ml), or Sendai virus (SDV, 10⁵ particles/ml) analyzed by RT-qPCR at 12 hrs post-treatment. Shown are mean±SD from 3 independent experiments for hPSC-derived macrophages and a single value for primary Kupffer cells/macrophages, after being normalized to values in corresponding vehicle control. (J). Representative immunofluorescent images of ALB (red) and A1AT (red) in hPSC-derived hepatocytes at day 20 post differentiation. Nuclei were stained with DAPI (scale bars, 100 μm). (K). Indocyanine green (ICG) staining of hPSC-derived hepatocytes at day 20 post differentiation. Left: representative image taken at 30 min post adding ICG; right: representative image taken at 12 hrs post removing ICG (scale bars, 100 μm). (L). Representative image of Periodic-acid-Schiff staining of hPSC-derived hepatocytes at day 20 post differentiation (bottom). Endoderm cells (END) at 5 days post differentiation were used as a negative control (scale bars, 100 μm). (M). Albumin (top) and urea (bottom) secretion of hPSC-derived hepatocytes at day 20 post differentiation. Endoderm cells (END) at 5 days post differentiation and primary human hepatocytes (PHH) were included as comparison. Shown are mean±SD from 3 independent experiments for hPSC-derived cells and from 2 independent experiments of two different PHH donors, after being normalized to endoderm (END) cells. (N). Representative bright-field images of hPSC-derived macrophages at 30 min (top) or 8 hrs (bottom) after being assembled into multicellular liver cultures (scale bars, 100 μm). (O)). Representative images of ASGR1/PCDH6 immunostaining of hPSC-derived hepatocytes and HSCs. The cell size was compared using ImageJ. (P). Purification of the individual type of cells from multicellular culture. Macrophages (Mac) were directly collected from the top compartment of the transwell system and subject to downstream analysis. To purify hepatocytes (HEP) and HSCs: cells from the bottom compartment were (i) treated with Versene and collagenase and dissociated into single-cell suspension; (ii) to separate hepatocytes from HSCs, single cells were blocked with 1% BSA, stained with anti-ASGR1 antibody, incubated with microbeads, and then separated by magnetic separator. Hepatocytes were collected from the fraction bound to the magnetic wall; HSCs were collected from the flow-through faction after another round of hepatocyte purification. Western blot analysis shows the purity of hepatocytes (indicated by ALB expression) and HSC (indicated by PDGFRB) fractions. RPS11 was used as a loading control. Statistical analysis was performed using a two-tailed unpaired t-test with Welch's corrections or One-way ANOVA/Tukey's multiple comparisons test to calculate exact p-values.

FIG. 10. Characterization of multicellular liver culture. (A). Transcript levels of PPARG were analyzed in purified HSCs from the hPSC-derived liver cultures with three-cell (HSC, HEP, and Mac), two-cell (HSC and HEP, HSC and Mac) and HSC monoculture at the indicated time points post co-culture by RT-qPCR. Shown are mean±SD from 4 independent experiments, after being normalized to FBS-activated HSCs. (B). Transcript levels of IL10 and macrophages marker CD163 were analyzed in purified macrophages from the hPSC-derived liver cultures with three-cell (HSC, HEP, and Mac) and two-cell (Mac and HEP, Mac and HSC) by RT-qPCR. Shown are mean±SD of delta Ct values from 3 independent experiments, after being normalized to housekeeping gene GAPDH. (C). Representative immunofluorescent images of in liver cultures showing lipid droplets (red), activated HSC marker COL1A1 (green) and hepatocyte marker ALB (blue), after being treated with LPS (100 ng/ml) for one week (scale bars, 100 μm). Enlarged images are shown for the select boxed area. (D). HSC activation markers FN and COL1A1 were analyzed by western blot in HSCs purified from three-cell (HEP, HSC and Mac) and two-cell (HSC and HEP, HSC and Mac) liver cultures treated with LPS (100 ng/ml) for one week. Differentiated HSCs without being co-cultured (week-0) were included as comparison. RPS11 was used as a loading control. (E). Representative immunofluorescent images of COL1A1 and ACTA2 in LPS (100 ng/ml)-induced activated HSCs. Nuclei were stained with DAPI (scale bars, 100 μm). Enlarged images are shown for the select boxed area. (F). Transcript levels of TGFB1 and IL1B in pooled cells from the liver cultures of three-cell (HSC, HEP, and Mac) and two-cell (HSC and HEP) were analyzed at one week post LPS (100 ng/ml) treatment by RT-qPCR. Shown are mean±SD from 4 independent experiments. (G). Transcript levels of IL1B and TGFB1 in purified individual cells from liver cultures treated with either vehicle control or LPS (100 ng/ml) for one week were analyzed by RT-qPCR. Fold changes were calculated by normalized to individual cells treated with vehicle control. Shown are mean±SD from 3 independent experiments. Statistical analysis was performed using a two-tailed unpaired t-test with Welch's corrections to or One-way ANOVA/Tukey's multiple comparisons test to calculate exact p-values.

FIG. 11. Modeling NAFLD in hPSC-derived multicellular liver cultures. (A). Representative immunofluorescent images of LD staining in hepatocytes purified from liver cultures treated with healthy or lipotoxic conditions for two weeks (scale bars, 100 μm). Enlarged images are shown for the select boxed area. (B-C). Transcript levels of lipogenic-associated enzyme genes ACC1, FASN, and SCD (B) and gluconeogenic-associated enzyme genes PCK1 and FBP1 (C) in hepatocytes purified from liver cultures treated with healthy (Heal) or lipotoxic (Lipo) conditions were analyzed by RT-qPCR at two weeks post-treatment. Shown are mean±SD from 4 independent experiments. (D-F). Hepatocytes were purified from liver cultures of three-cell (HEP, HSC and Mac), two-cell (HEP and Mac, HEP and HSC), or monocultured HEP under lipotoxic condition for two weeks and subject to quantification of oil-red (D), baseline glucose secretion (E), and insulin sensitivity (F). For oil-red quantification, shown are mean±SD from 6 randomly selected cells; for glucose secretion, shown are mean±SD from 4 independent experiments; for insulin sensitivity, analysis of glucose secretion (accumulation during 3 hrs) in the presence or absence of insulin (200 nM). Shown are mean±SD from 5 independent experiments. (G). Analysis of IL6 by ELISA in three-cell cultures (HEP, HSC and Mac), two-cell cultures (HSC and Mac, HSC and HEP), or HSC monocultures treated with lipotoxic condition for two weeks. Shown are mean±SD from 4 independent experiments. (H). Transcript levels of TLR4, CXCL10, TNF, and IL8 were analyzed in purified hepatocytes from liver cultures treated with healthy or lipotoxic condition for two weeks by RT-qPCR. Shown are mean±SD from 4 independent experiments. (I). Transcript levels of CXCL1, CXCL10, IL1B, and IL8 were analyzed in purified macrophages from liver cultures treated with healthy or lipotoxic condition for two weeks by RT-qPCR. Shown are mean±SD from 4 independent experiments. (J-O) hPSCs-derived multicellular liver cultures were treated with lipotoxic condition with either glucose (Lipo) or fructose (Fruct) for two weeks, transcript levels of SREBF1 and SCD (J), inflammatory cytokines IL-6 and IL1β (K), activation markers of HSCs (L), LDH activity (M), PARP AND Caspase-3 cleavages (N) and transcripts of BCL2L1 and CASP7 (O) were measured. Shown are mean±SD from 3 independent experiments. (P). Representative bright-field images of the bottom compartment of liver culture at 1 day (Day 1) or 14 days (Day 14) post lipotoxic condition exposure (scale bars, 100 μm). (Q). Representative immunofluorescent images (shown in grayscale) of HSCs in the bottom compartment of liver culture at 1 day (top, stained with anti-PCDH7) or 14 days (bottom, stained with anti-COL1A1) post lipotoxic condition exposure (scale bars, 100 μm). (R). hPSC- or primary cells of two donors (Pri-1 and Pri-2)-derived liver cultures were treated with healthy or lipotoxic condition for two weeks and purified macrophages were analyzed for transcript levels of IL1B and TNF by RT-qPCR. Shown are mean±SD from 3 independent experiments. Statistical analysis was performed using a two-tailed unpaired t-test with Welch's corrections or One-way ANOVA/Tukey's multiple comparisons test to calculate exact p-values.

FIG. 12. Generation and characterization of an isogenic pair of co-cultures harboring either PNPLA3WT or PNPLA3^{I148M}. (A). Representative immunofluorescent images of pluripotent markers OCT4 and SSEA4 staining in undifferentiated hPSC^{WT} and hPSC^{I148M} Nuclei were stained with DAPI (scale bars, 100 μm). (B). Transcript levels of lineage-specific marker genes LEFTY1 (endoderm), CDX2 (mesoderm), and PAX6 (ectoderm) were analyzed for trilineage differentiation of hPSC^{WT} and hPSC^{I148M} Shown are mean±SD from 3 independent experiments. (C). Indocyanine green (ICG) staining of hepatocytes derived from hPSC^{WT} and hPSC^{I148M} at day 20 post differentiation. Representative images were taken at 30 min post adding ICG (scale bars, 100 μm). (D). Representative image of Periodic-acid-Schiff staining of hepatocytes derived from hPSC^{WT} and hPSC^{I148M} at day 20 post differentiation (scale bars, 100 μm). (E-F). Transcript levels of P450 enzyme genes CYP3A4, CYP3A7, and CYP1A1 (E) and hepatocyte marker ASGR1 (F) in hepatocytes derived from hPSC^{WT} and hPSC^{I148M} at day 20 post differentiation were analyzed by RT-qPCR. Values were normalized to undifferentiated hPSCs. Shown are mean±SD from 3 independent experiments. (G). Transcript levels of cytokine genes CXCL10 and IL6 in macrophages derived from hPSC^{WT} and hPSC^{I148M} were analyzed at 12 hrs post-stimulation with defective influenza particles (10⁵ particles/ml). Shown are mean±SD from 3 independent experiments for hPSC-derived macrophages and a single value for primary Kupffer cells/macrophages, after being normalized to values in corresponding untreated control. (H). Transcript levels of PNPLA3 in hepatocytes and HSCs purified from liver cultures harboring either PNPLA3^{WT} or PNPLA3^{I148M} were analyzed by RT-qPCR at three weeks post-co-culture. Shown are mean±SD from 3 independent experiments. (I). Albumin secretion by hepatocytes purified from liver cultures harboring either PNPLA3WT or PNPLA3^{I148M} was analyzed by ELISA at the indicated time points post co-culture. Shown are mean±SD from 3 independent experiments. (J). Transcript levels of P450 enzyme genes CYP3A4, CYP3A7, and CYP1A1 in hepatocytes purified from liver cultures harboring either PNPLA3^{WT} or PNPLA3^{I148M} were analyzed by RT-qPCR at three weeks post co-culture. Shown are mean±SD from 3 independent, after being normalized to values in cells before being assembled into co-culture. (K-L). Transcript levels of IL6 in purified macrophages (K) and PPARG in purified HSCs (L) from liver cultures harboring either PNPLA3^{WT} or PNPLA3^{I148M} were analyzed by RT-qPCR at the indicated time points post co-culture. Shown are mean±SD from 3 independent experiments. Statistical analysis was performed using a two-tailed unpaired t-test with Welch's corrections or One-way ANOVA/Tukey's multiple comparisons to calculate exact p-values.

FIG. 13. Enhanced susceptibility to NAFLD phenotypes in co-cultures harboring PNPLA3^{I148M}. (A). Transcript levels of lipogenic-associated enzyme genes ACC1, SCD, and FASN in hepatocytes purified from the isogenic pair of liver cultures treated with lipotoxic conditions were analyzed by RT-qPCR at two weeks post-treatment. Shown are mean±SD from 4 independent experiments. (B). Analysis of baseline glucose secretion by hepatocytes purified from the isogenic pair of liver cultures treated with healthy condition at the indicated time points. Shown are mean±SD from 4 independent experiments, after being normalized to hepatocytes at day 0. (C). Transcript levels of PCK1 in hepatocytes purified from the isogenic pair of liver cultures treated with lipotoxic condition were analyzed at the indicated time points. Shown are mean±SD from 4 independent experiments, after being normalized to values at day 3 for each culture. (D). Hepatocytes purified for the experiment in (C) at the indicated time points were subject to western blot analysis of AKT and phosphorylated AKT (S463) up insulin (10 nM) stimulation for 10 min. Three replicates were included for both liver cultures. RPS11 was used as a loading control. (E-F). HSCs were purified from the isogenic pair of liver cultures treated with lipotoxic condition for two weeks and subject to analysis of HSC activation markers FN and ACTA2 by western blot (E), transcript level of quiescent marker PPARG by RT-qPCR (F, left), and retinol content by flow cytometry (F, right). For western blot analysis, two replicates were included and RPS11 was used as a loading control. For RT-qPCR and flow cytometry analysis, shown are mean±SD from 3 or 4 independent experiments. (G). Western blot analysis of fibronectin (FN) and two housekeeping markers ACTB and RPS11 were performed by using 5% of all attached cells for the experiment in FIG. 5E. (H-J). Individual cells were purified from the isogenic pair of liver cultures treated with lipotoxic condition for two weeks and subject to analysis of inflammatory markers IL6, TLR4, and TNF in hepatocytes (H), GM-CSF, CCL2, CCL5, and IL6 in HSCs (I), and IL1B, IL6, and TGFB1 in macrophages (J). Shown are mean±SD from 3 or 4 independent experiments. Statistical analysis was performed using a two-tailed unpaired t-test with Welch's corrections or One-way ANOVA/Tukey's multiple comparisons test to calculate exact p-values.

FIG. 14. Elevated IL6/STAT3 activity in PNPLA3^{I148M} co-cultures and in PNPLA3^{I148M} homozygous patients. (A). Individual cells were purified from the isogenic pair of liver cultures treated with lipotoxic condition for two weeks and transcript levels of STAT3 target genes BCL2, MMP14, and CCND1 were analyzed by RT-qPCR. Shown are mean±SD from 4 independent experiments. (B). Schematic showing variant calling workflow. From aligned sequence data from bulk RNA-seq experiments, bcftools was used to call variants similar to whole-genome sequencing approaches. Representative variant calling result tables are shown on the bottom; variant information on the SNP location is shown with reference (C) and input sequence (“.” If same as the reference sequence), along with quality scores and statistical significance. The top right table summarizes the number of patient samples with each genetic background (WT, I148M homozygous or heterogeneous) and with disease states (healthy control, NAFLD-early and NAFLD-moderate). (C). Heatmap summarizing normalized expression levels top variable differentially expressed genes as shown in FIG. 6C, comparing across PNPLA3 wild-type, I148M heterozygous and homozygous patients. Each line represents one patient sample, and the samples were grouped by conditions and genetic backgrounds as represented with the color bar annotations above. (D-E). Transcript levels of selected genes from the list of upregulated (D) and downregulated (E) genes in PNPLA3 I148M homozygous patients were analyzed by RT-qPCR in pooled cells (HEPs, HSCs and Macs) from the isogenic pair of liver cultures maintained in lipotoxic condition for two weeks. Shown are mean±SD from 4 independent experiments. (F). Heatmap summarizing average expression levels of IL6/STAT3 signaling pathway-related genes, across PNPLA3 wild-type, I148M heterozygous and homozygous patient samples. Statistical analysis was performed using a two-tailed unpaired t-test with Welch's corrections to calculate exact p-values.

FIG. 15. Elevated IL6/STAT3 activity mediates the enhanced susceptibility to NAFLD phenotypes in PNPLA3^{I148M} liver cultures (A). Analysis of IL6 by ELISA in the supernatant of shRNA control (Ctrl) or sh-IL6 liver cultures maintained in lipotoxic condition for two weeks. Shown are mean±SD from 3 independent experiments. (B). From the experiment in (A), the transcript levels of CHI3L1 and CXCL10 in purified macrophages were analyzed by RT-qPCR. Shown are mean±SD from 3 independent experiments. (C-D). From the experiment in (A), HSCs were purified and subject to western blot analysis of STAT3 and phosphorylated STAT3 (T705) (C), and to RT-qPCR analysis of the transcript levels of inflammatory cytokine CCL5 and quiescent marker PPARG (D). For western blot, RPS11 was used as a loading control. For RT-qPCR, shown are mean±SD from 3 independent experiments. (E-H). The isogenic pair of liver cultures were maintained in lipotoxic condition for two weeks in the presence of either isotype control antibody (Ctrl) or IL6 neutralizing antibody (nIL6 Ab, 100 ng/ml). Individual cells were purified from these liver cultures and subject to RT-qPCR analysis of lipogenic-associated enzyme genes ACC1 and FASN in hepatocytes (E) and inflammatory cytokine CHI3L1 in macrophages (F), western blot analysis of HSC activation markers FN, COL1A1, and ACTA2 (G), and phosphorylated STAT3 (H) in HSCs. For western blot, RPS11 was used as a loading control. For RT-qPCR, shown are mean±SD from 3 independent experiments. (I-J). PNPLA3^{WT} liver cultures were treated with either healthy or lipotoxic condition for 5 days and then IL6 (100 pg/ml) was added to one set of lipotoxic treated cultures for additional 2 days. Hepatocytes were purified and subject to analysis for triacylglycerol (TAG) level (I), transcript levels of ACC1 and FASN (J) by RT-qPCR. For TAG quantification, shown are mean±SD for 4 independent experiments; for RT-qPCR, shown are mean±SD from 3 independent experiments. Statistical analysis was performed using a two-tailed unpaired t-test with Welch's corrections or One-way ANOVA/Tukey's multiple comparisons test to calculate exact p-values.

FIG. 16. Elevated IL6/STAT3 activity induced by NF-κB activation in PNPLA3^{I148M} liver cultures. (A). Representative immunofluorescent images of phosphorylated p65 (green) in hPSC^{WT}-derived macrophages either untreated (mock) or treated with two supernatant (WT or I148M) were collected for cytokine array experiments in (FIG. 6A) for 1 hr. Cells were also stained with phalloidin (red) and nuclei were stained with DAPI (scale bars, 100 μm). Enlarged images are shown for the select boxed area. (B-C). hPSC^{WT}-derived macrophages were either untreated (mock) or treated with two supernatant (WT or I148M) collected for cytokine array experiments in (FIG. 6A) and subject to western blot analysis of IL6 induction (B) or RT-qPCR analysis of the selected NF-κB target genes (C) including ICAM1, MCP1 and TNF. For western blot, RPS11 was used as a loading control. For RT-qPCR, shown are mean±SD from 4 independent experiments. (D-E). hPSC^{WT}-derived macrophages were transduced with shRNA against MYD88 for 48 hrs, followed by treatment with the two supernatant (WT or I148M) for 12 hrs. Cells were then collected for RT-qPCR analysis of transcript levels of MYD88 (D) and IL6 (E). Shown are mean±SD from 3 or 4 independent experiments. Statistical analysis was performed using a two-tailed unpaired t-test with Welch's corrections or One-way ANOVA/Tukey's multiple comparisons test to calculate exact p-values.

### DETAILED DESCRIPTION

Provided herein are compositions, systems, kits, and methods for generating hPSC derived quiescent hepatic stellate cells (HSCs) and employing them in a liver model. In certain embodiments, methods and compositions are provided for maintaining the quiescent HSCs in a quiescent state for a time period.

A number of genetic polymorphisms have been associated with susceptibility to or protection against nonalcoholic fatty liver disease (NAFLD), but the underlying mechanisms remain unknown. Here, we focused on the rs738409 C>G single nucleotide polymorphism (SNP), which produces the I148M variant of patatin-like phospholipase domain-containing protein 3 (PNPLA3) and is strongly associated with NAFLD.

To enable mechanistic dissection, we developed a human pluripotent stem cells (hPSC)-derived multicellular liver culture by incorporating hPSC-derived hepatocytes, hepatic stellate cells, and macrophages. We first adopted this liver culture to model NAFLD by utilizing a lipotoxic milieu reflecting the circulating levels of disease risk factors in patients. We then created an isogenic pair of liver cultures differing only at rs738049 and compared NAFLD phenotypes development.

Our hPSC-derived liver culture recapitulated many key characteristics of NAFLD development and progression including lipid accumulation and oxidative stress, inflammatory response, and stellate cell activation. Under the lipotoxic conditions, the I148M variant caused the enhanced development of NAFLD phenotypes. These differences were associated with elevated IL6/STAT3 activity in liver cultures, consistent with transcriptomic data of liver biopsies from patients carrying the rs738409 SNP. Dampening IL6/STAT3 activity alleviated the I148M-mediated susceptibility to NAFLD, while boosting it in wild-type liver cultures enhanced NAFLD development. Finally, we attributed this elevated IL6/STAT3 activity in liver cultures carrying the rs738409 SNP to increased NF-kB activity. Our study thus reveals a potential causal link between elevated IL6/STAT3 activity and 148M-mediated susceptibility to NAFLD.

In certain embodiments, the media employed herein comprises at least 5 . . . 10 . . . 15 . . . or all 20 of the reagents below in Table 1.

TABLE 1
Supplement Composition

| | |
|---|---|
| 1) | Catalase |
| 2) | Glutathione reduced |
| 3) | NO INSULIN (B27 normally has insulin) |
| 4) | Superoxide |
| 5) | Human Holo-Transferin |
| 6) | T3 |
| 7) | L-carnitine |
| 8) | Ethanolamine |
| 9) | D+-galactose |
| 10) | Putrescine |
| 11) | Sodium selenite |
| 12) | Corticosterone |
| 13) | Linoleic acid |
| 14) | Linolenic acid |
| 15) | Progesterone |
| 16) | Retinolacetate |
| 17) | DL-alphatocopherol |
| 18) | DL-alpha tocopherol |
| 19) | Oleic acid |
| 20) | Pipecolicacid |
| 21) | Biotin |

### EXAMPLES

### Example 1

### The IL6/STAT3 Axis Dictates the PNPLA3-Mediated Susceptibility to Nonalcoholic Fatty Liver Disease

PNPLA3 I148M genetic variant increases the risk of NAFLD development and progression. To explore the underlying mechanism, we developed a hPSC-derived liver culture to study NAFLD by utilizing disease risk factors derived from patients. In liver cultures containing the variant, we observed that I148M variant increased the risk of NAFLD by elevating IL6/STAT3 signaling, which agreed with analysis of patient liver biopsies. Our findings demonstrate a potential causal link between elevated IL6/STAT3 activity and I148M variant-mediated risk of NALFD. Further, our liver culture is a useful platform for exploring genetic variants in NAFLD development.

Here, we developed a multicellular liver culture system that utilizes human pluripotent stem cell (hPSC)-derived hepatocytes, HSCs, and macrophages, three cell types involved in NAFLD. Under conditions reflecting NAFLD-associated metabolic dysfunction [6, 7], this system recapitulates disease phenotypes. After generating an isogenic hPSC pair expressing rs738409, we found a pivotal role for the signal transducer and activator of transcription 3 (STAT3) and interleukin 6 (IL6) pathway in PNPLA3-mediated NAFLD susceptibility.

### Results

Generation of a hPSC-Derived Multicellular Liver Culture

To establish an in vitro human-relevant hepatic system that recapitulates liver complexity, we co-cultured hPSC-derived hepatocytes, HSCs, and liver-resident macrophages (Kupffer cells). However, few methods robustly produce quiescent HSCs [12], whose activation into a proliferative fibrogenic state is critical in NAFLD progression [1].

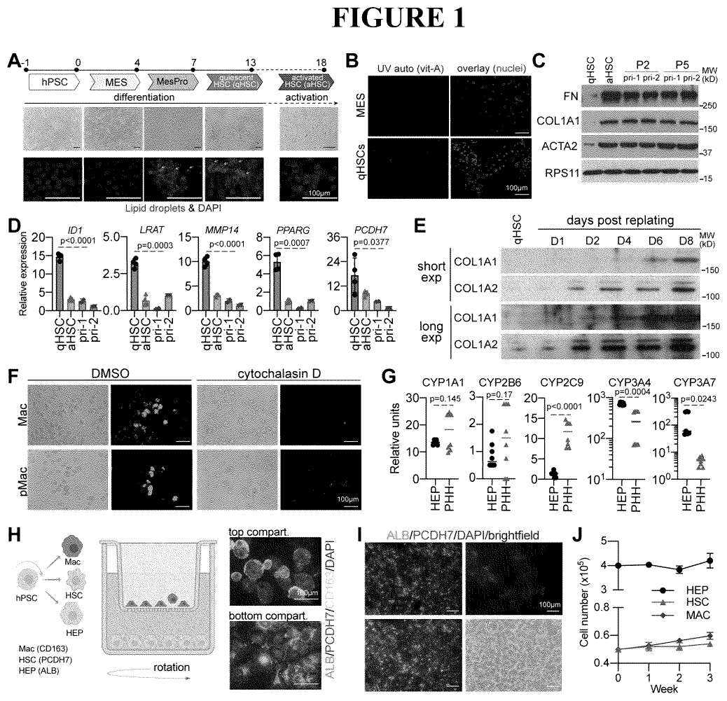

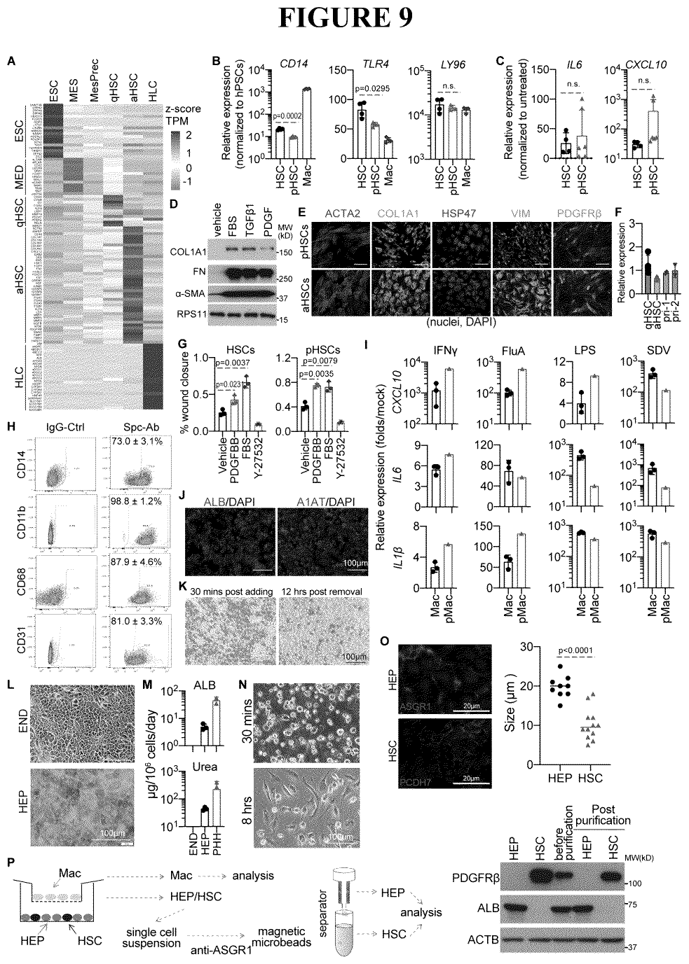

Thus, we developed a differentiation protocol for quiescent hPSC-derived HSCs (hPSC-HSCs) using fibroblast growth factor 1 (FGF1) and FGF2, epidermal growth factor (EGF), retinol and lipids, which are important for HSC quiescence (FIG. 1A) [13]. After thirteen days, differentiated progeny expressed HSC markers (FIG. 9A). Vitamin A storage in lipid droplets, a feature of quiescence, was observed in hPSC-HSCs (FIG. 1B) and lost upon activation-inducing replating after day 13 (FIG. 1A). Compared to primary HSCs (pHSCs) that are rapidly activated in culture, hPSC-HSCs at day 13 exhibited markedly lower levels of activation markers collagen type I alpha 1 chain (COL1A1), fibronectin (FN) and a-Smooth muscle actin (α-SMA) (FIG. 1C), and higher expression of quiescence markers (FIG. 1D and FIG. 9A).

Another hallmark of quiescent HSCs is their ability to respond to inflammatory and pro-fibrotic stimuli like lipopolysaccharide (LPS) and transforming growth factor beta 1 (TGFβ1). hPSC-HSCs expressed LPS receptors (CD14/TLR4/LY96) and induced cytokine upon stimulation (FIG. 9B-C). HSCs activated by pro-fibrotic stimuli differentiate into myofibroblasts and secrete extracellular matrix (ECM) components like collagen and FN. Upon re-plating, collagen secretion by activated hPSC-HSCs reached significantly higher levels in 7 days (FIG. 1E). Pro-fibrotic stimuli like fetal bovine serum (FBS), TGFβ1, and platelet-derived growth factor (PDGF) also clearly activated hPSC-HSCs (FIG. 9D). Activated hPSC-HSCs could be cultured for up to 3 passages without affecting marker expression (FIG. 9E-F). Functionally, hPSC-HSCs activated by FBS and TGFβ1 showed enhanced motility similar to pHSCs that was markedly inhibited by Y-27632, an inhibitor of p160-Rho-associated coiled-coil kinase (ROCK) (FIG. 9G). Altogether, this protocol generates quiescent hPSC-HSCs that are phenotypically and functionally similar to pHSCs upon activation.

We also compared hPSC-derived macrophages (hPSC-macrophages) and hepatocytes (hPSC-hepatocytes) to primary human Kupffer cells and hepatocytes (PHHs). hPSC-macrophages expressed characteristic Kupffer cell markers (FIG. 9H), and showed phagocytic capacity as measured by ingestion of dead E. coli (FIG. 1F). After treatment with LPS, defective influenza virus or Sendai viral particles, both Kupffer cells and hPSC-macrophages transcribed inflammatory cytokines (FIG. 9I). Thus, hPSC-macrophages recapitulate the critical inflammatory and antimicrobial functions of Kupffer cells.

The majority of hPSC-hepatocytes expressed hepatocyte-specific markers albumin (ALB) and α1-antitrypsin (A1AT) (FIG. 9J). Functionally, hPSC-hepatocytes resembled PHHs as seen by indocyanine green uptake, glycogen synthesis and ALB/urea secretion (FIG. 9K-M). Many cytochrome P450 (CYP) enzyme activities were comparable between hPSCs-hepatocytes and PHHs other than CYP2C9 and CYP3A7 activity (FIG. 1G), which suggests an immature state. Collectively, hPSC-hepatocytes and macrophages reproduce many key functions of their primary counterparts.

To establish a liver culture system, we co-cultured hPSC-hepatocytes, HSCs and macrophages at an 8:1:1 ratio, proportions reflective of healthy livers [14]. We used a transwell to separate macrophages from co-cultured hepatocytes and HSCs while permitting diffusion of secreted factors (FIG. 1H) and used a horizontally shaking platform to promote medium flow. After one day, hepatocytes (ALB⁺) and HSCs (PCDH7⁺) spontaneously clustered as ˜3-6 HSCs surrounded by a larger number of hepatocytes (FIG. 1H-I) while macrophages remained adherent to the transwell membrane (FIG. 9N). Importantly, cellular morphology, size and spatial patterning remained stable for at least three weeks without proliferation (FIG. 1J and FIG. 9O), reminiscent of a healthy liver. To deconvolute cell type-specific responses within the liver culture, we developed a pipeline to isolate each cell type through immunomagnetic cell separation using asialoglycoprotein receptor 1 (ASGR1) expression (FIG. 9P). Altogether, we have established a stable self-organizing multicellular “liver culture” of hPSC-derived hepatocytes, HSCs, and macrophages. This system performs comparably using both human embryonic stem cells (HUES8) and induced pluripotent stem cells (iPSC-W3).

### Characterizing the Multicellular Liver Culture

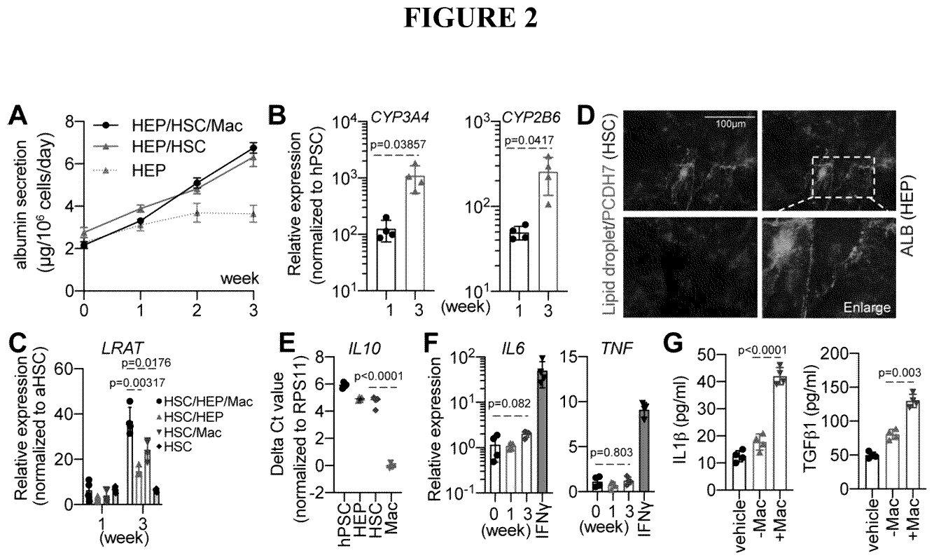

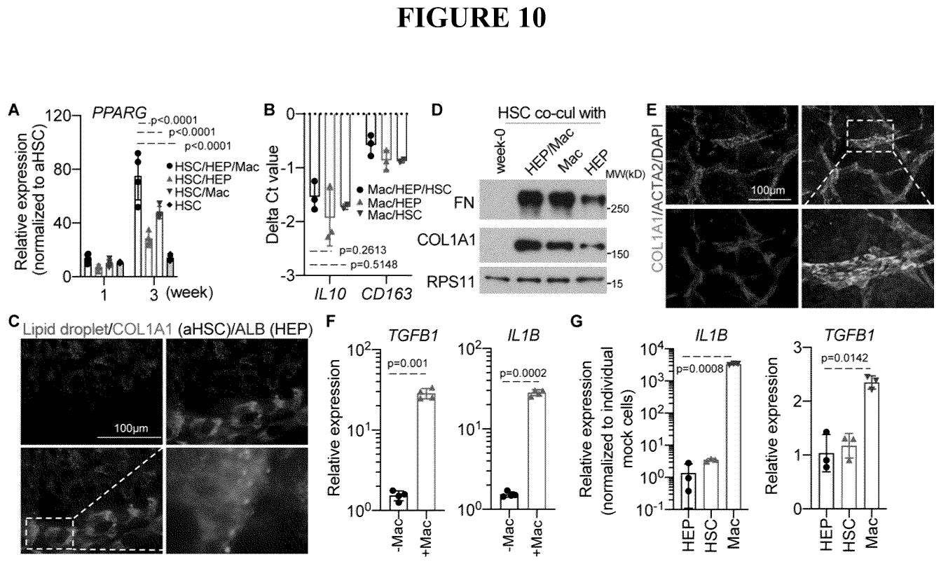

Liver function depends on intercellular communication mediated by soluble factors and cell-cell contact. We first determined how cell type-defining properties were affected by co-culture. In hepatocytes, albumin secretion increased in the liver culture and HEP/HSC co-culture (FIG. 2A), suggesting that HSCs promote hepatocyte maturation. This is consistent with the upregulation of CYP3A4 and CYP2B6, which are involved in drug oxidation (FIG. 2B). In HSCs, quiescence markers lecithin-retinol acyltransferase (LRAT) and peroxisome proliferator-activated receptor γ (PPARG) were increased in liver culture relative to two-cell co-cultures and HSC monoculture (FIG. 2C and FIG. 10A). The presence of lipid droplets further confirmed quiescence (FIG. 2D). Within liver cultures, macrophages expressed the highest level of IL10 (FIG. 2E), which was minimally affected by removing hepatocytes or HSCs (FIG. 10B). Thus, macrophages may establish a stable anti-inflammatory environment in liver culture, which is consistent with the absence of inflammatory cytokines (FIG. 2F) [15]. Altogether, the multicellular liver culture facilitates intercellular interactions important for a homeostatic liver-like state.

Next, we examined how this homeostatic state was altered by LPS, which is implicated in chronic liver diseases [16]. After one week of LPS exposure, HSCs lost cytoplasmic lipid droplets and expressed elevated activation markers (FIG. 10C-E), Interestingly, LPS-induced HSC activation was significantly reduced without macrophages (FIG. 10D). To dissect the macrophage contribution, we examined proinflammatory cytokine production. With LPS treatment, liver cultures produced much higher levels of IL1β and TGFβ1 (FIG. 2G) derived mainly from macrophages (FIG. 10F-G). Macrophages may therefore exacerbate liver disease by secreting cytokines that promote HSC activation [1, 11].

### Modeling NAFLD in Multicellular Liver Culture

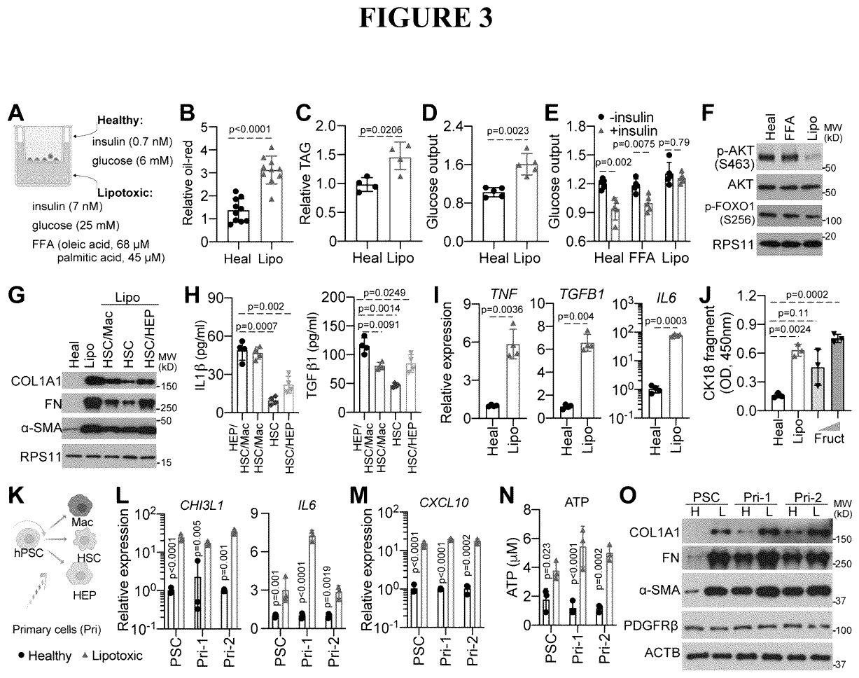

To model NAFLD, we applied a lipotoxic milieu composed of glucose (25 mM), insulin (7 nM), palmitic acid (45 μM), and oleic acid (68 μM). The levels of these risk factors mimic circulating concentrations in NAFLD patients [6, 7]. For comparison, “healthy” conditions contained normal physiological levels of insulin (700 pM) and glucose (6 mM) (FIG. 3A).

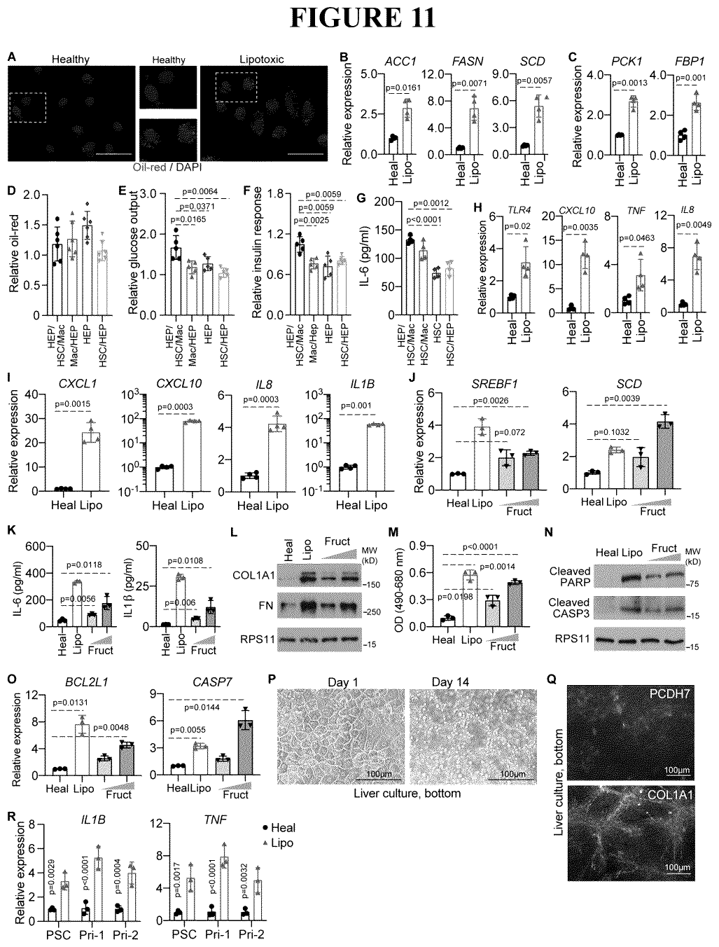

First, we assessed hepatocyte lipid accumulation. Limited amounts were seen in purified hepatocytes from healthy cultures, but lipids accumulated ˜3-fold under lipotoxic conditions (FIG. 3B and FIG. 11A), Triacylglycerol (TAG) levels also increased by ˜1.5-fold (FIG. 3C). The expression of lipogenesis-associated enzymes in hepatocytes was upregulated, consistent with patient data [1, 11] (FIG. 11B). Thus, the lipotoxic environment recapitulates alterations in hepatocyte lipid metabolism seen in NAFLD.

Since NAFLD is often accompanied by insulin resistance, we next evaluated hepatocyte glucose homeostasis. Under lipotoxic conditions, we found a 2-fold increase in glucose secretion and gluconeogenesis-associated enzyme expression like phosphoenolpyruvate carboxykinase 1 (PCK1) and fructose-1,6-bisphosphatase 1 (FBP1) (FIG. 3D and FIG. 11C). Since insulin inhibits gluconeogenesis by decreasing PCK1 transcription [17], we directly tested insulin resistance. Hepatocytes from lipotoxic cultures showed a markedly attenuated glucose output to insulin challenge (FIG. 3E). AKT and FOXO1 phosphorylation, regulators of insulin signaling, were also significantly decreased (FIG. 3F). Increased FOXO1 activity, the main transcription factor of PCK1 [17], eventually led to PCK1 upregulation and insulin resistance (FIG. 11C).

We then evaluated whether HSCs and macrophages affected hepatocyte phenotypes. Similar lipid accumulation was observed between liver culture and hepatocyte monoculture with lipotoxic exposure (FIG. 11D). In contrast, insulin responsiveness decreased in two-cell co-cultures compared to liver cultures (FIG. 11E-F). Thus, macrophages and HSCs contribute variably to hepatocyte NAFLD phenotypes (FIG. 11E), which are both autonomous and non-autonomous.

Next, we evaluated how NAFLD risk factors affected HSC activation. Under lipotoxic conditions, quiescent HSCs differentiated into activated myofibroblasts that highly expressed COL1A1 and α-SMA (FIG. 3G). Activation was decreased in the absence of hepatocytes, macrophages, or both cell types (FIG. 3G), indicating a joint contribution to HSC activation under lipotoxic conditions. Inflammatory cytokines upregulated in NAFLD, such as TGFβ1, IL6, and IL1β, were much higher in the supernatants of two- and three-cell co-cultures compared to HSC monocultures (FIG. 3H and FIG. 11G). This was confirmed at the transcript level in purified hepatocytes and macrophages (FIG. 3I and FIG. 11H-I). Since fructose also contributes to NAFLD pathogenesis [1], we replaced glucose with fructose in the lipotoxic milieu and observed the induction of similar NAFLD disease phenotypes (FIG. 11J-L). Hence, the liver culture recapitulates key characteristics of NAFLD progression.

We then assessed hepatocyte death in liver cultures given its importance in NAFLD progression [1]. Under lipotoxic conditions, culture supernatants showed elevated caspase-generated cytokeratin 18 (CK18) fragments (FIG. 3J), a biomarker of NAFLD severity [18], and lactate dehydrogenase (LDH) activity (FIG. 11M). Purified hepatocytes displayed apoptotic signs like upregulation of poly (ADP-ribose) polymerase 1 (PARP1) and caspase 3 (CAPS3) cleavage (FIG. 11N-O)).

Beyond biochemical changes, we also monitored cellular morphology. Hepatocytes formed lipid droplets that gradually led to cytoplasmic vacuolation (FIG. 11P). After 2-3 weeks, some hepatocytes swelled and became rounded. For HSCs, most gained a myofibroblast-like shape within two weeks (FIG. 11Q). In contrast, macrophage morphology remained largely unchanged. Cells strongly adhered to the transwell for one week before some began to detach or remained loosely attached to the membrane.

Finally, we compared how liver cultures from hPSCs and primary cells responded to lipotoxic conditions (FIG. 3K). The lipotoxic milieu triggered similar inflammatory phenotypes in hPSC-macrophages and primary Kupffer cells (FIG. 3L and FIG. 11R), including chitinase-3-like protein 1 (CHI3L1) and IL6 expression [19-21]. We observed similar parallels between PHHs and hPSC-hepatocytes in terms of chemokine expression and intracellular ATP levels (FIG. 3M-N), hallmarks of oxidative stress in NAFLD [1]. Finally, we compared HSC activation between primary and hPSC-HSCs. Lipotoxic conditions induced activation of both HSCs (FIG. 30), but hPSC-HSCs maintained a more quiescent state under healthy conditions as described in FIG. 1. The parallels between hPSC-derived and primary liver cultures support the physiological relevance of our model.

Generation of an Isogenic Pair of Co-Cultures Harboring Either PNPLA3^{WT} or PNPLA3^{I148M}

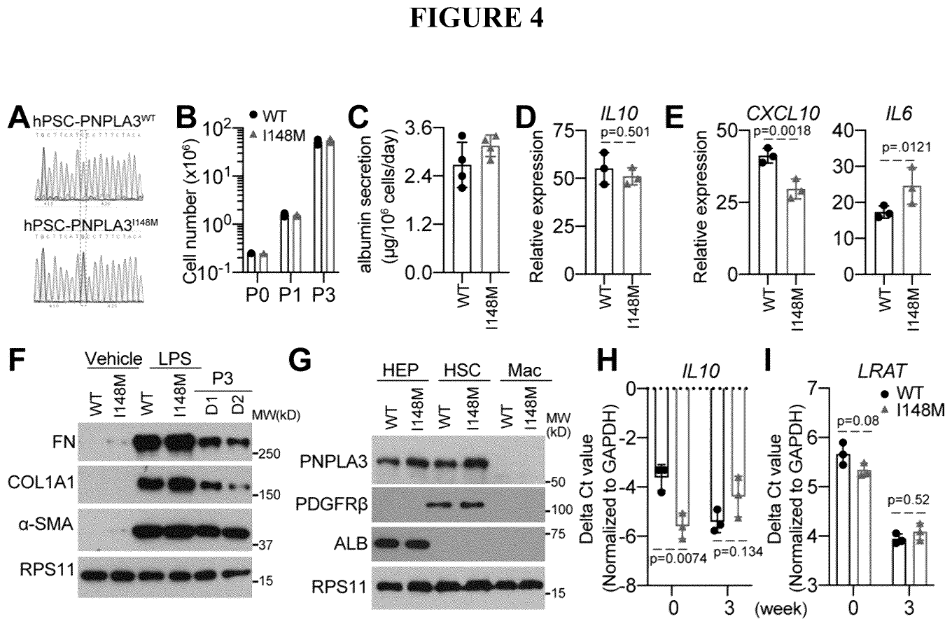

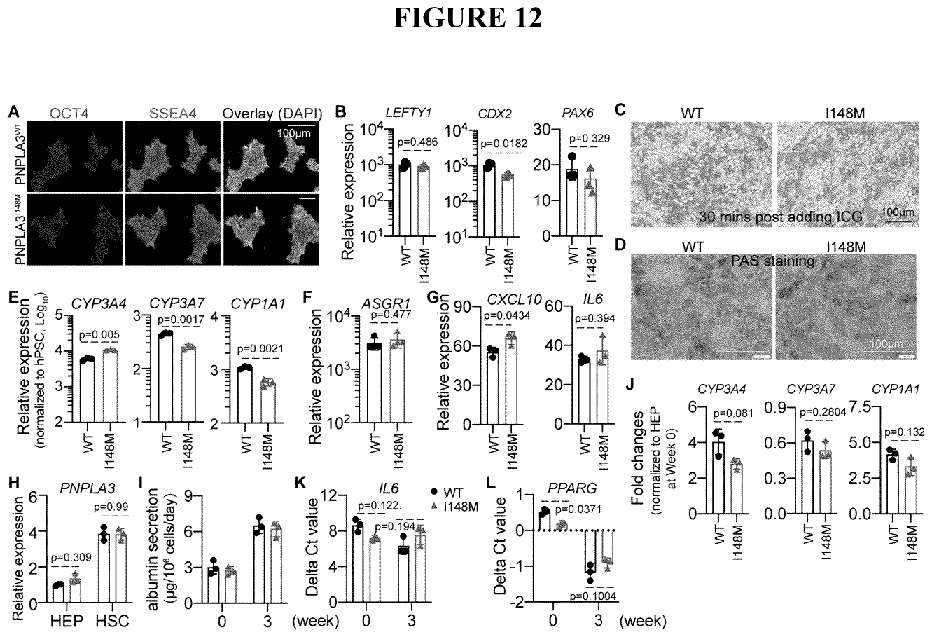

To analyze genetic variants, we generated an isogenic pair of hPSCs harboring wildtype or I148M PNPLA3. We introduced the rs738409 C>G point mutation into hPSC^{WT} cells to make a homozygous hPSC^{I148M} line (FIG. 4A) whose in vitro proliferation and trilineage differentiation were nearly identical (FIG. 4B and FIG. 12A-B).

We first evaluated cell-intrinsic changes associated with the I148M variant. hPSC^{WT} and hPSC^{I148M}-derived hepatocytes showed comparable albumin secretion (FIG. 4C), indocyanine green uptake, glycogen synthesis and maturity (FIG. 12C-E). Importantly, ASGR1 expression was similar between hepatocyte backgrounds, which ensures comparable purification efficiencies (FIG. 12F). hPSC^{WT} and hPSC^{I148M}-derived macrophages showed a tolerogenic phenotype with similar baseline IL10 expression (FIG. 4D), and comparable inflammatory cytokine induction to LPS or defective influenza particles (FIG. 4E and FIG. 12G). Quiescent HSC differentiation efficiency was comparable between hPSC^{WT} and hPSC^{I148M} (FIG. 4F). Activation markers were lower in both genetic backgrounds than pHSCs and comparably induced after LPS treatment (FIG. 4F). Overall, we did not observe cell-intrinsic differences in hPSC^{I148M}-derived hepatocytes, macrophages and HSCs.

We then compared PNPLA3^{WT} and PNPLA3^{I148M} liver cultures under healthy conditions. In both cases, cellular morphology and patterning remained stable for at least 3 weeks. In our liver cultures, PNPLA3 was detected in HSCs and hepatocytes, but not macrophages (FIG. 4G) as previously reported [22]. Protein expression of the I148M variant was higher than WT (FIG. 4G) despite similar transcript levels (FIG. 12H). This result is consistent with findings that the I148M variant is resistant to ubiquitination and proteasomal degradation [23]. We then examined cellular phenotypes after 3 weeks of co-culture. Purified hepatocytes were indistinguishable between genetic backgrounds in terms of albumin secretion and CYP family enzyme expression (FIG. 12I-J). Macrophages expressed similar levels of cytokines IL10 (FIG. 4H) and IL6 (FIG. 12K). Finally, HSC quiescence markers were comparable (FIG. 4I and FIG. 12L). With an isogenic pair of hPSC-derived liver cultures, we were now poised to dissect how the PNPLA3 I148M variant contributed to NAFLD.

### Co-Cultures Harboring PNPLA3^{I148M} Exhibit Enhanced Susceptibility to NAFLD Risk Factors

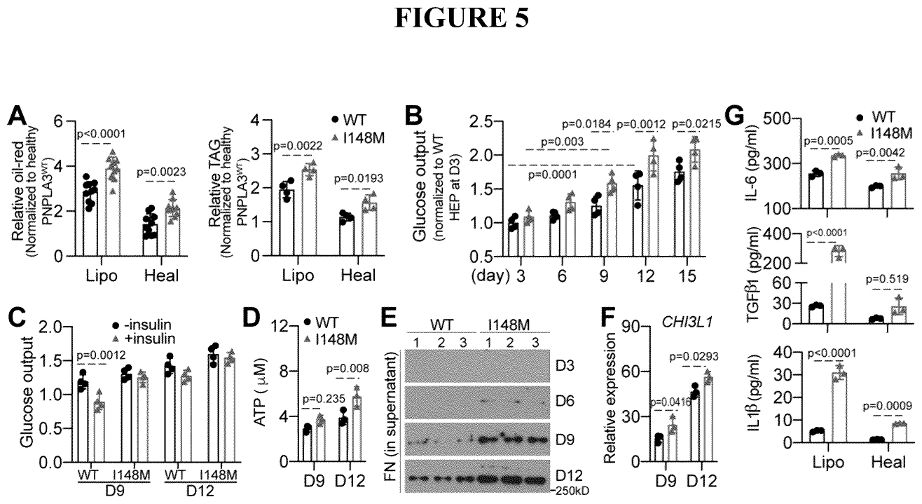

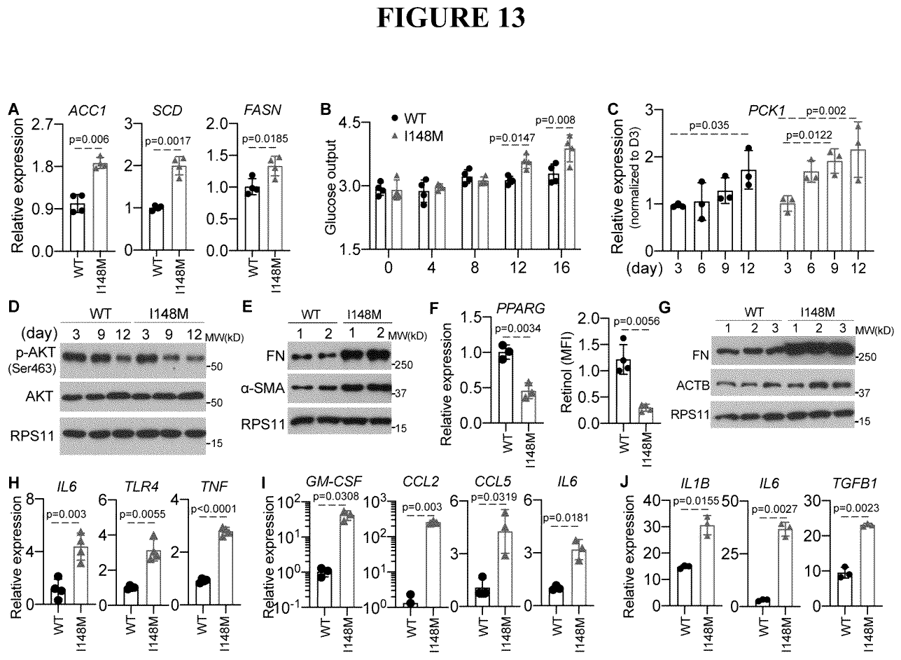

To study the I148M variant in NAFLD development, we compared the response of PNPLA3^{WT} or PNPLA3^{I148M} liver cultures after two-weeks in lipotoxic conditions. We quantified hepatocyte lipid accumulation and observed a 1.5-fold greater increase in oil-red staining and intracellular TAG levels in PNPLA3^{I148M} hepatocytes compared to PNPLA3^{WT} (FIG. 5A), consistent with the severe steatosis associated with rs738409 [4]. Higher transcript levels of lipogenic-associated enzymes further support this observation (FIG. 13A). Interestingly, lipid staining and TAG levels were elevated in PNPLA3^{I148M} hepatocytes in liver cultures compared to PNPLA3^{WT} cultures under healthy conditions (FIG. 5A). This baseline suggests that the I148M variant promotes hepatocyte lipid accumulation, which matches published in vitro and clinical findings [8].

Next, we examined hepatocyte glucose regulation. Under healthy conditions, glucose secretion was comparable between both liver cultures until day 12, after which an increase was evident in PNPLA3^{I148M} liver cultures (FIG. 13B). This difference was accelerated under lipotoxic conditions and evident in PNPLA3^{I148M} cultures by day 9 (FIG. 5B). At this point, PNPLA3^{I148M} hepatocytes lost their responsiveness to insulin whereas PNPLA3^{WT} hepatocytes remained sensitive (FIG. 5C). Insulin resistance correlated with elevated ATP (FIG. 5D), increased PCK1, and diminished AKT phosphorylation upon insulin stimulation (FIG. 13C-D). Thus, hepatocytes in PNPLA3^{I148M} liver cultures are more susceptible to glucose dysregulation than their WT counterparts.

Focusing on HSCs, we found that HSCs in both liver cultures developed fibroblast-like morphologies. However, expression of activation markers was higher in HSCs purified from PNPLA3^{I148M} liver cultures while retinol content and quiescence marker PPARG were lower (FIG. 13E-F). Similar to hepatocytes, monitoring fibronectin secretion showed that HSCs from PNPLA3^{I148M} liver cultures were activated with faster kinetics than PNPLA3^{WT} cultures (FIG. 5E and FIG. 13G). Thus, PNPLA3^{I148M} HSCs show enhanced and accelerated activation under prolonged lipotoxic insult.

Given the inflammatory nature of NAFLD, we found that inflammatory cytokine levels were higher in both the supernatants and separately purified cells from PNPLA3^{I148M} than PNPLA3^{WT} liver cultures (FIG. 5F-G and FIG. 13H-J). Since macrophages do not express PNPLA3 (FIG. 4G), this upregulation likely arises from crosstalk with hepatocytes and/or HSCs. Collectively, the PNPLA3^{I148M} genetic background under lipotoxic conditions enhances NAFLD phenotypes including steatosis, HSC activation and inflammation.

### Elevated IL6/STAT3 Activity in PNPLA3^{I148M} Liver Cultures and NAFLD Patient

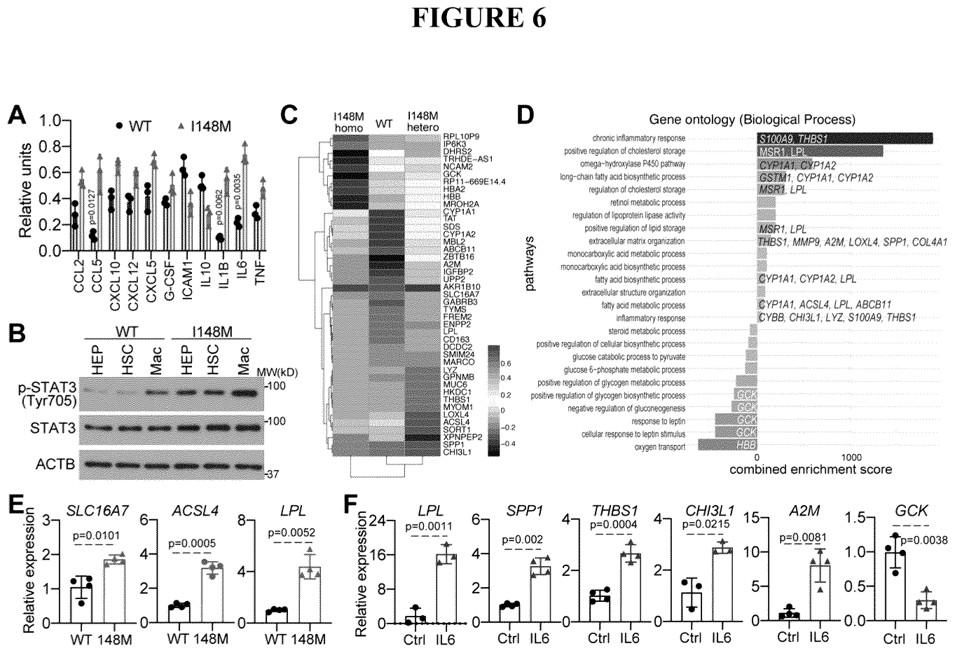

Our in vitro results indicate that the PNPLA3 I148M variant causally enhances NAFLD susceptibility as suggested by genome-wide association studies [4]. Since lipotoxic conditions induce strong inflammation in PNPLA3^{I148M} liver cultures, a defining feature of NAFLD progression [11], we hypothesized that secreted factors in the culture supernatant likely drives the enhancement of NAFLD phenotypes. To identify potential soluble effectors, we used a human cytokine array to analyze supernatants from day 6 of lipotoxic exposure since hepatocyte and HSC differences appeared after ˜1 week (FIG. 6A). Numerous cytokines and chemokines were elevated in PNPLA3^{I148M} supernatants including CXCL5, CXCL10, CXCL12, CCL5, IL1β, IL6 and TNFα (FIG. 6A).

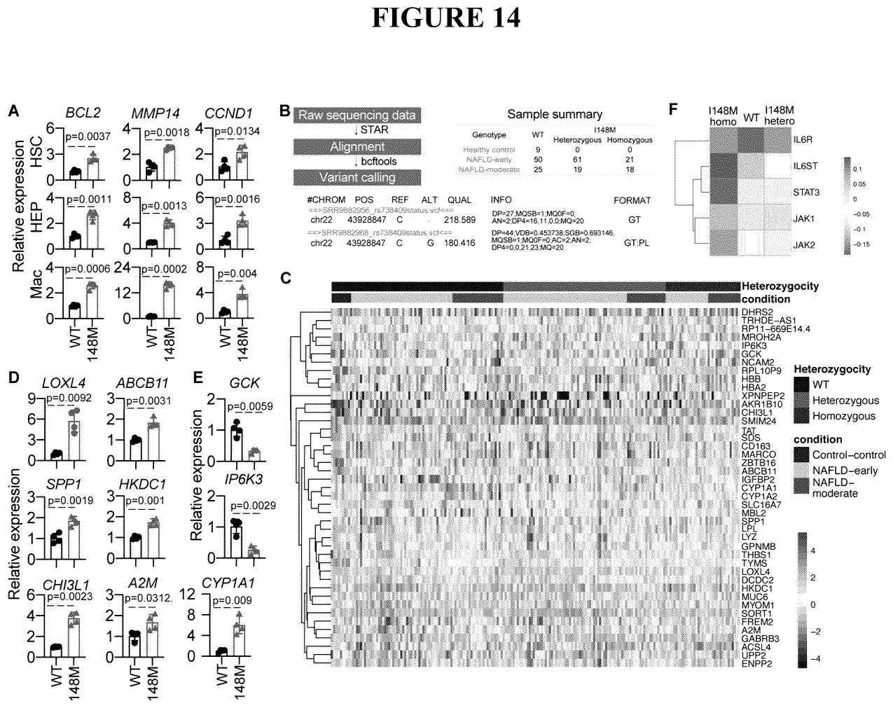

The three most differentially expressed cytokines and chemokines were CCL5, IL6 and IL1β, which have all been implicated in NAFLD pathogenesis [11]. Interestingly, IL6 is significantly increased in the liver of NAFLD patients and associated with severity [21]. IL6 binds to the IL6R/gp130 receptor, which signals via the JAK1/JAK2/STAT3 pathway, to promote STAT3 phosphorylation and target gene transcription. Compared to PNPLA3^{WT} liver cultures, all three purified cell types from the PNPLA3^{I148M} cultures showed elevated STAT3 phosphorylation (FIG. 6B). This general enhancement of IL6/STAT3 signaling under lipotoxic conditions was supported by higher expression of STAT3 target genes (FIG. 14A).

These in vitro results led us to hypothesize that increased IL6/STAT3 signaling could also be found in patients harboring the rs738409 C>G SNP. By genotyping transcriptomic data of NAFLD and healthy liver biopsies [9, 24], we classified patients as wild-type, I148M homozygous or I148M heterozygous (FIG. 14B). The transcriptional profiles of wild-type and I148M heterozygous biopsies were similar and distinct from I148M homozygous patients (FIG. 6C and FIG. 14C). Gene ontology (GO) analysis of genes upregulated in the I148M homozygous group mapped to regulation of cholesterol storage, omega-hydroxylase P450, monocarboxylic biosynthesis, and lipoprotein lipase (FIG. 6D). This enrichment in pathways related to fatty acid and lipid metabolism is consistent with differential metabolic activity noted in PNPLA3^{I148M} patients [25]. To compare directly with our liver cultures, we quantified gene expression on co-cultures without cell separation and focused on genes that contributed to the GO analysis relevant to liver function (FIG. 6D and Table 2). The general patterns of higher (e.g., solute carrier family 16 member 7 (SLC16A7) and lipoprotein lipase (LPL)) and lower expression (i.e., glucokinase (GCK)) between PNPLA3^{I148M} and PNPLA3^{WT} liver cultures (FIG. 6E and FIG. 14D-E) were similar to the liver biopsy results (FIG. 6C).

TABLE 2
Upregulated and downregulated GOs

| | | | | | |
|---|---|---|---|---|---|
| | | Adjusted | | Combined | |
| Term | P-value | P-value | Odds Ratio | Score | Genes |

GO_Upregulated_DEGs

| | | | | | |
|---|---|---|---|---|---|
| extracellular matrix organization | 6.80E−07 | 5.54E−04 | 10.47387131 | 148.7371817 | VCAN; GSN; VCAM1; COL4A1; SPP1; |
| (GO:0030198) | | | | | LOXLA; A2M; MMP9; THBS1 |
| phagocytosis, engulfment | 6.99E−06 | 0.002847926 | 38.28763829 | 454.5204059 | MSR1; MARCO; GSN; THBS1 |
| (GO:0006911) | | | | | |
| extracellular structure organization | 8.53E−05 | 0.013929908 | 9.23793911 | 86.55087338 | VCAN; VCAM1; COL4A1; SPP1; |
| (GO:0043062) | | | | | MMP9; THBS1 |
| external encapsulating structure | 8.75E−05 | 0.013929908 | 9.193691244 | 85.90252504 | VCAN; VCAM1; COL4A1; SPP1; |
| organization (GO:0045229) | | | | | MMP9; THBS1 |
| chronic inflammatory response | 1.10E−04 | 0.013929908 | 204.4102564 | 1863.514013 | S100A9; THBS1 |
| (GO:0002544) | | | | | |
| monocarboxylic acid metabolic | 1.18E−04 | 0.013929908 | 11.56790556 | 104.6478988 | CYP1A2; CYP1A1; LPL; |
| process (GO:0032787) | | | | | ACSL4; ABCB11 |
| plasma membrane invagination | 1.37E−04 | 0.013929908 | 34.55902778 | 307.4876947 | MSR1; MARCO; GSN |
| (GO:0099024) | | | | | |
| long-chain fatty acid biosynthetic | 1.37E−04 | 0.013929908 | 34.55902778 | 307.4876947 | GSTM1; CYP1A2; CYP1A1 |
| process (GO:0042759) | | | | | |
| positive regulation of cholesterol | 1.64E−04 | 0.01486931 | 153.3 | 1335.739374 | MSR1; LPL |
| storage (GO:0010886) | | | | | |
| neutrophil degranulation | 1.99E−04 | 0.01486931 | 5.578528685 | 47.55537042 | FCN1; GSN; CYBB; CHI3L1; KCNAB2; |
| (GO:0043312) | | | | | LYZ; MMP9; S100A9 |
| neutrophil activation involved in | 2.10E−04 | 0.01486931 | 5.53061152 | 46.83609789 | FCN1; GSN; CYBB; CHI3L1; |
| immune response (GO:0002283) | | | | | KCNAB2; LYZ; MMP9; S100A9 |
| neutrophil mediated immunity | 2.19E−04 | 0.01486931 | 5.49519774 | 46.30658813 | FCN1; GSN; CYBB; CHI3L1; KCNAB2; |
| (GO:0002446) | | | | | LYZ; MMP9; S100A9 |
| retinoid metabolic process | 2.58E−04 | 0.016143536 | 14.31818182 | 118.3322454 | AKR1B10; CYP1A2; CYP1A1; LPL |
| (GO:0001523) | | | | | |
| positive regulation of epithelial cell | 2.80E−04 | 0.016278468 | 13.99858907 | 114.5370551 | RAB25; ENPP2; MMP9; THBS1 |
| migration (GO:0010634) | | | | | |
| steroid metabolic process | 4.11E−04 | 0.022341542 | 12.59238095 | 98.17581003 | CYP51A1; CYP1A2; |
| (GO:0008202) | | | | | CYP1A1; ABCB11 |
| complement activation, lectin | 4.89E−04 | 0.023440071 | 76.63461538 | 584.2073646 | FCN1; MBL2 |
| pathway (GO:0001867) | | | | | |
| omega-hydroxylase P450 pathway | 4.89E−04 | 0.023440071 | 76.63461538 | 584.2073646 | CYP1A2; CYP1A1 |
| (GO:0097267) | | | | | |
| regulation of cysteine-type | 5.96E−04 | 0.026998883 | 68.11623932 | 505.7478274 | GSN; MMP9 |
| endopeptidase activity involved in | | | | | |
| apoptotic signaling pathway | | | | | |
| (GO:2001267) | | | | | |
| fatty acid metabolic process | 7.98E−04 | 0.03422751 | 10.48306878 | 74.78070586 | CYP1A1; LPL; ACSL4; ABCB11 |
| (GO:0006631) | | | | | |
| monocarboxylic acid transport | 9.24E−04 | 0.037646173 | 17.25607639 | 120.5678573 | SLC16A7; SLC6A11; ABCB11 |
| (GO:0015718) | | | | | |
| steroid catabolic process | 9.80E−04 | 0.038041667 | 51.07948718 | 353.8653505 | CYP1A2; SPP1 |
| (GO:0006706) | | | | | |
| inflammatory response | 0.001041675 | 0.038589334 | 7.063799283 | 48.50658 | CYBB; CHI3L1; LYZ; |
| (GO:0006954) | | | | | S100A9; THBS1 |
| monocarboxylic acid biosynthetic | 0.001235982 | 0.043704842 | 15.52578125 | 103.9589199 | SDS; LPL; ABCB11 |
| process (GO:0072330) | | | | | |
| regulation of cholesterol storage | 0.001287014 | 0.043704842 | 43.77802198 | 291.3615902 | MSR1; LPL |
| (GO:0010885) | | | | | |
| cellular component disassembly | 0.0014141 | 0.043933523 | 14.78422619 | 97.00318176 | GSN; A2M; MMP9 |
| (GO:0022411) | | | | | |
| extracellular matrix disassembly | 0.0014141 | 0.043933523 | 14.78422619 | 97.00318176 | GSN; A2M; MMP9 |
| (GO:0022617) | | | | | |
| negative regulation of viral entry | 0.001455466 | 0.043933523 | 40.8574359 | 266.8982937 | FCN1; GSN |
| into host cell (GO:0046597) | | | | | |
| positive regulation of macrophage | 0.001633865 | 0.047557151 | 38.30192308 | 245.7760373 | MSR1; LPL |
| derived foam cell differentiation | | | | | |
| (GO:0010744) | | | | | |
| fatty acid biosynthetic process | 0.001745309 | 0.049049214 | 13.69370404 | 86.96629614 | CYP1A2; CYP1A1; LPL |
| (GO:0006633) | | | | | |
| epoxygenase P450 pathway | 0.001822144 | 0.049077884 | 36.04705882 | 227.3755291 | CYP1A2; CYP1A1 |
| (GO:0019373) | | | | | |
| skeletal system development | 0.001952545 | 0.049077884 | 8.154607297 | 50.87350831 | VCAN; EVC; ZBTB16; CHI3L1 |
| (GO:0001501) | | | | | |
| negative regulation of viral life cycle | 0.002020236 | 0.049077884 | 34.04273504 | 211.2195434 | FCN1; GSN |
| (GO:1903901) | | | | | |
| positive regulation of cell migration | 0.002075913 | 0.049077884 | 6.00836999 | 37.11582982 | GPNMB; RAB25; ENPP2; |
| (GO:0030335) | | | | | MMP9; THBS1 |
| regulation of lipoprotein lipase | 0.002228076 | 0.049077884 | 32.24939271 | 196.9346887 | SORT1; LPL |
| activity (GO:0051004) | | | | | |
| retinol metabolic process | 0.002228076 | 0.049077884 | 32.24939271 | 196.9346887 | CYP1A2; CYP1A1 |
| (GO:0042572) | | | | | |
| positive regulation of lipid storage | 0.002228076 | 0.049077884 | 32.24939271 | 196.9346887 | MSR1; LPL |
| (GO:0010884) | | | | | |
| positive regulation of smoothened | 0.002228076 | 0.049077884 | 32.24939271 | 196.9346887 | EVC; DCDC2 |
| signaling pathway (GO:0045880) | | | | | |
| positive regulation of epithelial cell | 0.002672736 | 0.056980766 | 29.17509158 | 172.85228 | GSN; THBS1 |
| apoptotic process (GO:1904037) | | | | | |
| long-chain fatty acid metabolic | 0.002726687 | 0.056980766 | 11.63261719 | 68.68674222 | CYP1A2; CYP1A1; ACSL4 |
| process (GO:0001676) | | | | | |

GO_Dowmregulated_DEGs

| | | | | | |
|---|---|---|---|---|---|
| positive regulation of cellular biosynthetic | 0.001600497 | 0.106063576 | 14.58879882 | 93.91452979 | EGR1; HBB; GCK |
| process (GO:0031328) | | | | | |
| positive regulation of cell death | 0.003312017 | 0.106063576 | 25.92447917 | 148.0339044 | EGR1; HBB |
| (GO:0010942) | | | | | |
| cellular response to heparin (GO:0071504) | 0.006483689 | 0.106063576 | 199.7 | 1006.181577 | EGR1 |
| positive regulation of tau-protein kinase | 0.007775574 | 0.106063576 | 159.752 | 775.8783981 | EGR1 |
| activity (GO:1902949) | | | | | |
| response to heparin (GO:0071503) | 0.007775574 | 0.106063576 | 159.752 | 775.8783981 | EGR1 |
| regulation of hormone biosynthetic process | 0.007775574 | 0.106063576 | 159.752 | 775.8783981 | EGR1 |
| (GO:0046885) | | | | | |
| steroid metabolic process (GO:0008202) | 0.00802358 | 0.106063576 | 16.23529412 | 78.34130973 | UGT2B17; DHRS2 |
| oxygen transport (GO:0015671) | 0.009065844 | 0.106063576 | 133.12 | 626.0954814 | HBB |
| positive regulation of hormone metabolic | 0.009065844 | 0.106063576 | 133.12 | 626.0954814 | EGR1 |
| process (GO:0032352) | | | | | |
| regulation of ketone biosynthetic process | 0.009065844 | 0.106063576 | 133.12 | 626.0954814 | EGR1 |
| (GO:0010566) | | | | | |
| inhibitory postsynaptic potential | 0.009065844 | 0.106063576 | 133.12 | 626.0954814 | CHRNA4 |
| (GO:0060080) | | | | | |
| cellular response to leptin stimulus | 0.011641548 | 0.106063576 | 99.83 | 444.5604443 | GCK |
| (GO:0044320) | | | | | |
| response to leptin (GO:0044321) | 0.011641548 | 0.106063576 | 99.83 | 444.5604443 | GCK |
| regulation of tau-protein kinase activity | 0.012926986 | 0.106063576 | 88.73333333 | 385.8514207 | EGR1 |
| (GO:1902947) | | | | | |
| myeloid dendritic cell differentiation | 0.012926986 | 0.106063576 | 88.73333333 | 385.8514207 | DHRS2 |
| (GO:0043011) | | | | | |
| inositol phosphate biosynthetic process | 0.014210816 | 0.106063576 | 79.856 | 339.6876147 | IP6K3 |
| (GO:0032958) | | | | | |
| gas transport (GO:0015669) | 0.01549304 | 0.106063576 | 72.59272727 | 302.5203443 | HBB |
| negative regulation of gluconeogenesis | 0.016773662 | 0.106063576 | 66.54 | 272.011885 | GCK |
| (GO:0045721) | | | | | |
| renal absorption (GO:0070293) | 0.018052682 | 0.106063576 | 61.41846154 | 246.5620209 | HBB |
| positive regulation of glycogen biosynthetic | 0.018052682 | 0.106063576 | 61.41846154 | 246.5620209 | GCK |
| process (GO:0045725) | | | | | |
| myeloid dendritic cell activation | 0.018052682 | 0.106063576 | 61.41846154 | 246.5620209 | DHRS2 |
| (GO:0001773) | | | | | |
| positive regulation of glycogen metabolic | 0.019330102 | 0.106063576 | 57.02857143 | 225.0399736 | GCK |
| process (GO:0070875) | | | | | |
| electron transport chain (GO:0022900) | 0.019330102 | 0.106063576 | 57.02857143 | 225.0399736 | DHRS2 |
| glucuronate metabolic process (GO:0019585) | 0.019330102 | 0.106063576 | 57.02857143 | 225.0399736 | UGT2B17 |
| positive regulation of vasoconstriction | 0.020605924 | 0.106063576 | 53.224 | 206.6249708 | AVPR1A |
| (GO:0045907) | | | | | |
| regulation of systemic arterial blood pressure | 0.020605924 | 0.106063576 | 53.224 | 206.6249708 | AVPR1A |
| by hormone (GO:0001990) | | | | | |
| neuron cell-cell adhesion (GO:0007158) | 0.020605924 | 0.106063576 | 53.224 | 206.6249708 | NCAM2 |
| polyol biosynthetic process (GO:0046173) | 0.020605924 | 0.106063576 | 53.224 | 206.6249708 | IP6K3 |
| regulation of steroid biosynthetic process | 0.021880151 | 0.106063576 | 49.895 | 190.7074424 | EGR1 |
| (GO:0050810) | | | | | |
| cellular response to zinc ion (GO:0071294) | 0.023152783 | 0.106063576 | 46.95764706 | 176.8256072 | MT1E |
| regulation of protein sumoylation | 0.023152783 | 0.106063576 | 46.95764706 | 176.8256072 | EGR1 |
| (GO:0033233) | | | | | |
| synaptic transmission, cholinergic | 0.024423824 | 0.106063576 | 44.34666667 | 164.6235293 | CHRNA4 |
| (GO:0007271) | | | | | |
| cellular glucuronidation (GO:0052695) | 0.024423824 | 0.106063576 | 44.34666667 | 164.6235293 | UGT2B17 |
| cellular response to copper ion | 0.024423824 | 0.106063576 | 44.34666667 | 164.6235293 | MT1E |
| (GO:0071280) | | | | | |
| regulation of protein modification by small | 0.025693274 | 0.106063576 | 42.01052632 | 153.8226357 | EGR1 |
| protein conjugation or removal | | | | | |
| (GO:1903320) | | | | | |
| hydrogen peroxide catabolic process | 0.026961136 | 0.106063576 | 39.908 | 144.2019251 | HBB |
| (GO:0042744) | | | | | |
| sensory perception of pain (GO:0019233) | 0.026961136 | 0.106063576 | 39.908 | 144.2019251 | CHRNA4 |
| negative regulation of small molecule | 0.028227412 | 0.106063576 | 38.00571429 | 135.5839309 | GCK |
| metabolic process (GO:0062014) | | | | | |
| exogenous drug catabolic process | 0.028227412 | 0.106063576 | 38.00571429 | 135.5839309 | CYP2D7 |
| (GO:0042738) | | | | | |

*For both lists, only top 40 GOs are shown.

Beyond metabolism, GO terms related to inflammatory response were also highly enriched, consistent with the inflammatory signature of PNPLA3^{I148M} liver cultures (FIG. 6D). Many STAT3 target genes were also upregulated in I148M homozygous biopsies (Table 3).

TABLE 3
Known STAT3 target genes within the DEGs list

| GENENAME | baseMean | log2FoldChange |
|---|---|---|
| CCND1 | 1609.407766 | 0.281718679 |
| BCL2 | 61.05551744 | 0.272841567 |
| MMP14 | 303.9091067 | 0.285650516 |
| MMP9 | 20.98366036 | 0.836690327 |
| MMP19 | 35.65286359 | 0.347314909 |
| MMP24 | 23.72129287 | 0.634087368 |
| SERPINA11 | 4635.174472 | 0.285430757 |
| NOTCH2 | 1695.284146 | 0.236776097 |
| PSD3 | 1593.515767 | 0.342667962 |
| DHRS9 | 43.0970123 | 0.324487523 |
| FOS | 57.26590328 | −1.215037773 |
| MMP1 | 1.071902475 | −1.743638629 |
| MMP28 | 5.551249109 | −0.654918208 |
| GADD45B | 456.6233007 | −0.429136459 |

Expression of IL6 signaling components also showed relatively higher levels in this patient subset (FIG. 14F). Of note, IL6 expression was low across all samples and genetic backgrounds, which may be due to technical limitations in transcript detection as IL6 is elevated in the serum and livers of NAFLD patients [21]. Importantly, many of the genes in I148M homozygous patients that drive the GO results (FIG. 6D and Table 2) were readily regulated by IL6 treatment in our liver cultures (FIG. 6F), indicating the importance of IL6 signaling.

Together, these analyses comparing our in vitro liver cultures with primary liver biopsies suggest a strong association of elevated IL6/STAT3 activity with the PNPLA3 I148M variant.

### Elevated IL6/STAT3 Activity Mediates Enhanced Susceptibility to NAFLD Phenotypes in PNPLA3^{I148M} Liver Cultures

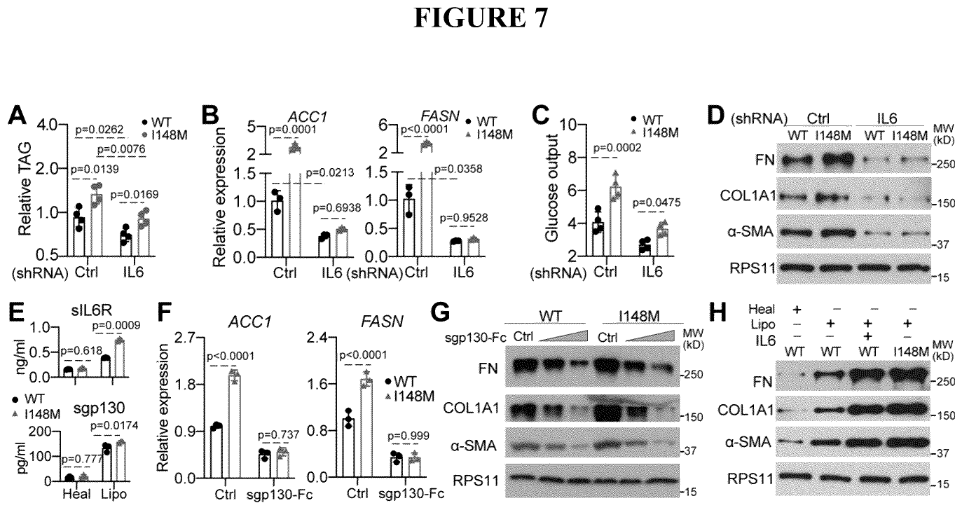

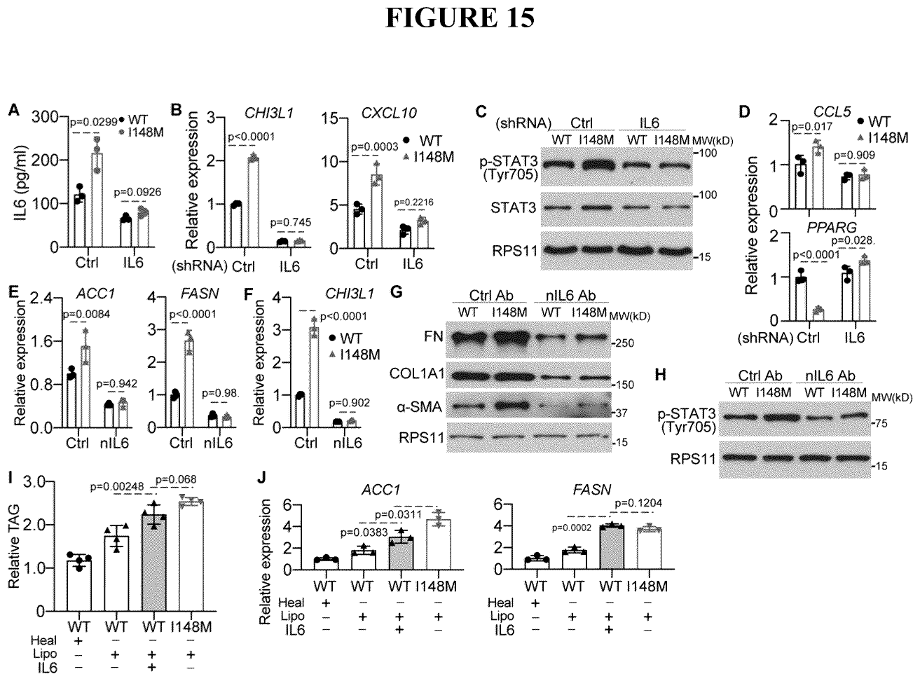

We therefore sought to test directly whether elevated IL6/STAT3 activity enhances susceptibility to NAFLD phenotypes. To downregulate IL6, we transduced shRNA against IL6 or control (termed sh-IL6 and sh-Ctrl) after forming liver cultures to minimize effects on differentiation. IL6 levels in PNPLA3^{I148M} liver cultures under lipotoxic conditions were reduced to similar levels as PNPLA3^{WT} liver cultures (FIG. 15A). The higher TAG levels in PNPLA3^{I148M} hepatocytes similarly normalized to PNPLA3^{WT} culture levels (FIG. 7A). Lipogenesis-associated enzyme expression substantially decreased in both liver cultures (FIG. 7B), suggesting that downregulating IL6 inhibits hepatocyte lipogenesis. The elevated hepatocyte glucose secretion in PNPLA3^{I148M} was also markedly diminished by IL6 knockdown (FIG. 7C), indicating that elevated IL6 signaling contributes to the observed PNPLA3^{I148M} glucose dysregulation. In macrophages, inflammatory responses were also significantly reduced by IL6 downregulation (FIG. 15B). Finally, IL6 knockdown significantly reduced HSC activation, and nearly normalized the degree of activation between PNPLA3^{I148M} and PNPLA3WT liver cultures under lipotoxic conditions (FIG. 7D). This normalization was also seen in purified HSCs when measuring STAT3 phosphorylation, inflammatory cytokine CCL5 and quiescence marker PPARG (FIG. 15C-D). Finally, we found that treating liver cultures with an IL6 neutralizing antibody to block IL6/STAT3 signaling produced similar changes to IL6 knockdown in hepatocytes, macrophages, and HSCs (FIG. 15E-H).

IL6 signaling mainly occurs through two distinct pathways: (i) classical-signaling through membrane-bound IL6R leading to gp130 homodimerization, and (ii) trans-signaling through metalloproteinase-cleaved soluble IL6R (sIL6R) producing the IL6/sIL6R complex that signals through gp130 [26-28]. Thus, we sought to distinguish between these possibilities given the importance of IL6 signaling. Levels of sIL6R and soluble gp130 (sgp130) were increased under lipotoxic conditions and reached slightly higher levels in PNPLA3^{I148M} cultures (FIG. 7E). Upon selective inhibition of trans-signaling with sgp130 Fc-protein (sgp130-Fc), we observed decreases in lipogenesis-associated enzyme expression and HSC activation (FIG. 7F-G). At a higher sgp130-Fc concentration (100 ng/ml), the degree of HSC activation under lipotoxic conditions was nearly normalized between PNPLA3^{I148M} and PNPLA3^{WT} cultures, similar to IL6 knockdown (FIG. 7D and FIG. 15G), indicating the importance of IL6 trans-signaling.

As a complementary approach, we determined if IL6 was sufficient to enhance NAFLD susceptibility in lipotoxic conditions by supplementing IL6 in PNPLA3^{WT} cultures to a level similar to that of PNPLA3^{I148M} cultures. We began after 5 days of lipotoxic exposure when disease phenotypes begin to manifest (FIG. 5B) and only maintained liver cultures for 2 more days to minimize the compounded effect of continuous IL6 secretion on our observations. Supplementing IL6 increased intracellular TAG levels and lipogenesis-associated enzyme expression in PNPLA3^{WT} hepatocytes (FIG. 15I-J) to levels comparable to PNPLA3^{I148M} liver cultures. Changes in glucose secretion were not detected, however, likely due to the brevity of IL6 supplementation. Moreover, HSCs from PNPLA3^{WT} cultures were dramatically activated by IL6 addition (FIG. 7H). Collectively, these results indicate that elevated IL6/STAT3 activity drives the enhanced susceptibility to NAFLD phenotypes in PNPLA3^{I148M} liver cultures.

### Elevated IL6/STAT3 Activity Induced by NF-κB Activation in PNPLA3^{I148M} Liver Cultures

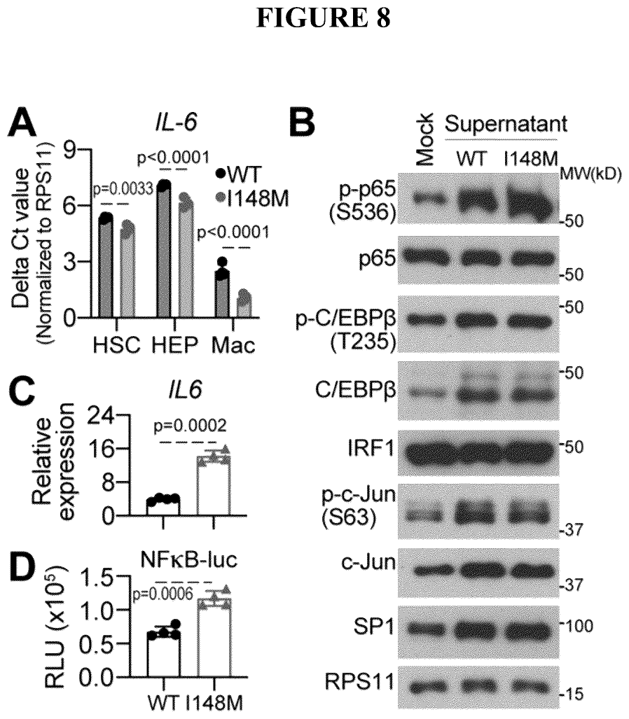

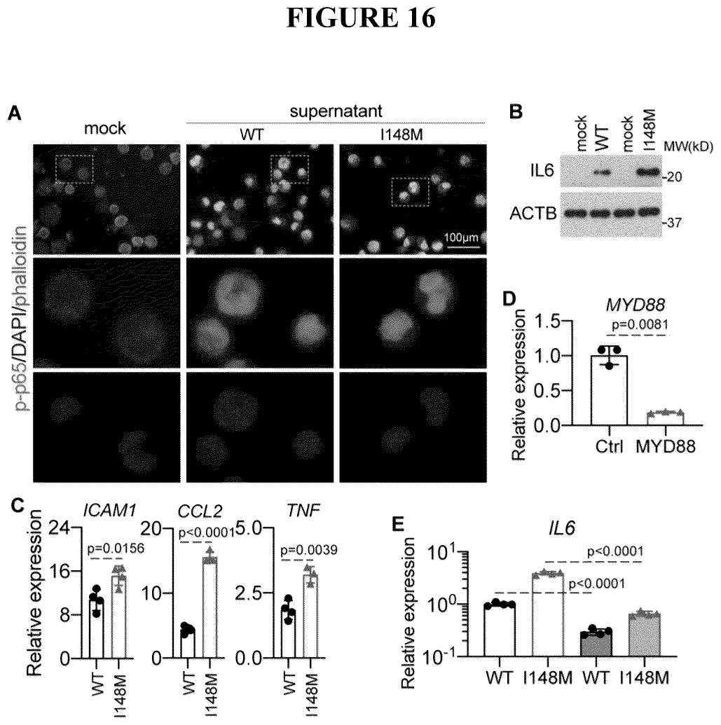

Finally, we sought to understand the basis of differential IL6 expression in PNPLA3^{WT} and PNPLA3^{I148M} liver cultures. IL6 is primarily induced under inflammatory conditions by activated stromal and immune cells [27]. By comparing the purified cells from PNPLA3^{WT} and PNPLA3^{I148M} cultures, the elevated IL6 expression comes primarily from macrophages (FIG. 8A). The IL6 promoter includes binding sites for activator protein 1 (AP1/c-Jun), interferon regulatory factor 1 (IRF1), CCAAT-enhancer-binding protein β (C/EBPβ), specificity protein 1 (SP1) and NF-κB [20]. As positive feedback is known to regulate IL6 production, we treated macrophages with the day 6 lipotoxic culture supernatants (FIG. 6A) in the presence of IL6 neutralizing antibodies. By examining activation of the aforementioned transcription factors, we found that phosphorylation of p65, the active form of NF-κB, was induced at a slightly higher level by PNPLA3^{I148M} liver culture supernatant relative to PNPLA3^{WT} (FIG. 8B). We further confirmed that supernatants induced nuclear translocation of phosphorylated p65 (FIG. 16A). The PNPLA3^{I148M} liver culture supernatant significantly induced IL6 and NF-κB target genes (FIG. 8C and FIG. 16B-C), consistent with enhanced NF-κB reporter luciferase expression (FIG. 8D). Knockdown of myeloid differentiation factor 88 (MYD88), an upstream adaptor of NF-κB signaling, in macrophages reduced IL6 induction by culture supernatants (FIG. 16D-E). Therefore, the NF-κB pathway likely mediates the elevated IL6 production seen in PNPLA3^{I148M} liver cultures and the resulting IL6/STAT3 signaling enhances susceptibility to NAFLD.

Current in vitro models of NAFLD are insufficient for dissecting how genetic variants produce disease phenotypes. Immortalized cells show metabolic and immune alterations whereas primary cells are limited by availability and donor-to-donor variability. In particular, combining primary cells of unmatched backgrounds complicates variant-to-function inference [18, 29]. While hPSCs-derived liver organoids are an attractive renewable platform, they lack mesenchymal components involved in NAFLD [11]. Thus, there is a clear unmet need for new disease models.

Addressing these limitations, we established a multicellular liver culture. Notably, we developed differentiation conditions that generated quiescent HSCs in contrast to the activated phenotypes of HSC lines and pHSCs. The multicellularity is important for studying proteins expressed in multiple cell-types like PNPLA3 [22]. Finally, we developed a protocol to purify individual cell types to deconvolute their separate or joint contributions to disease. To model NAFLD, we applied a lipotoxic milieu reflecting disease-associated metabolic dysfunction and showed clear recapitulation of features like abnormal lipid accumulation, insulin resistance and inflammation. The amount of steatosis and ECM deposition suggest that we are modeling early NAFLD, consistent with a recent primary cell model [18]. This model is therefore positioned to understand how disease-associated SNPs influence the NAFLD development. By comparing an isogenic liver cultures, we found that PNPLA3^{I148M} liver cultures developed stronger NAFLD phenotypes with accelerated kinetics, consistent with increased disease susceptibility.

In PNPLA3^{I148M} liver cultures, we observed enhanced IL6/STAT3 signaling that was consistent with transcriptomics from patient liver biopsies. IL6 is a pleiotropic cytokine involved in tissue homeostasis, regeneration and metabolism [20, 27]. Hepatocyte-specific ablation exaggerates diet-induced inflammation [30], yet IL6 hyperactivation is implicated in inflammatory disease pathogenesis [26-28]. IL6 signals via distinct pathways: classic-signaling mediates protective and regenerative effects whereas trans-signaling is often pro-inflammatory. In our liver cultures, globally blocking IL6 signaling not only reduced NALFD development, but also nearly normalized the differences between PNPLA3^{WT} and PNPLA3^{I148M} Selective inhibition of trans-signaling produced similar results. Thus, blocking IL6 trans-signaling may be a therapeutic candidate for rs738409-homozygous NAFLD patients and a clinical trial of sgp130-Fc (Olamkicept) in inflammatory bowel disease indicates that this strategy is anti-inflammatory and well-tolerated [31]. Since IL6 trans-signaling and the I148M variant have been respectively associated with HCC development and risk [11, 32], our liver platform is a promising pre-clinical tool for variant-to-function studies for liver diseases beyond NAFLD.

Macrophages are major source of IL6 in our liver cultures, yet PNPLA3 expression is low or undetectable. Thus, inflammation driven by the I148M variant is likely downstream soluble factors from hepatocytes and/or HSCs. Several possible identities for these factors merit future investigation: (i) Metabolic lipid products from the enzymatic activity of PNPLA3 in hepatocytes and HSCs could promote metabolic stress. (ii) Hepatocyte-derived biomolecules whose secretion is modulated by TAG accumulation. Oxidized low-density lipoprotein cholesterol, for instance, is increased in NAFLD patients and associated with IL6 production [33]. Alternatively, damaged hepatocytes could release damage-associated molecular patterns (DAMPs) that drive immune activation [1]. (iii) Macrophage-derived products like lipoprotein-associated phospholipase A2 (Lp-PLA2) known to be elevated in NAFLD could produce IL6 feedback activation in myeloid cells [35]. Such possibilities highlight the importance of multicellularity in disease models to understand intercellular communication.

In summary, we have developed an hPSC-derived model of NAFLD suitable for mechanistic dissection of genetic variants. As proof-of-concept, we show that the strong association between the rs738409 C>G SNP in PNPLA3 and NAFLD susceptibility is caused by elevated IL6/STAT3 activity that accelerates disease development. Our liver culture is therefore a useful platform to conduct causal variant-to-function studies in NAFLD.

### Abbreviations

SNP: single nucleotide polymorphisms; NAFLD: non-alcoholic fatty liver disease; hPSC: human pluripotent stem cells; HCC: hepatocellular carcinoma; HSC: hepatic stellate cells; FGF: fibroblast growth factor; EGF: epidermal growth factor; MES: mesoderm; TAG: triacylglycerol; LPS: lipopolysaccharide; GO: gene oncology; ECM: extracellular matrix; LDL: low-density lipoprotein cholesterol; ER: endoplasmic reticulum; ACC1: acetyl-CoA carboxylase alpha; FASN: fatty acid synthase; SCD: stearoyl-CoA desaturase; CHI3L1: chitinase-3-like protein 1; CXCL10: C—X—C motif chemokine ligand 10; ASGR1: Asialoglycoprotein Receptor 1; TNF: tumor necrosis factor; DEG: differentially expressed genes

### MATERIALS AND METHODS

### Cell Culture Reagents

Primary human hepatocytes, Kupffer cells, hepatic stellate cells were maintained in Lonza cell growth media according to the manufacturer's instructions.

### Stem Cell Maintenance and Differentiation

Human pluripotent stem cells (hPSCs) including ESCs (HUES8) and iPSCs (iPSC-W3) were cultured on growth factor-reduced matrigel according to the manufacturer's recommendations in feeder-independent mTeSR1-based medium (regular or mTeSR1 Plus). Cultures were replenished with fresh medium every day. Cells were passaged every 4-6 days as clumps using ReLeSR. For all the experiments in this study, hES/iPSCs were used between passage 30 and 40. For experiments reported in FIGS. 1 to 3, both HUES8 and iPSC-W3 were tested; however, only data collected from iPSC-W3 were shown in these figures. For experiments reported in FIGS. 4 to 7, only HUES8 was used. Experiments with hESCs were performed at Rockefeller University.

Differentiation of hPSC-Derived Quiescent Hepatic Stellate Cells (HSC)

For HSC differentiation, cell culture plates were coated twice with matrigel to increase the thickness of the protein membrane. Human PSCs were first dissociated into single-cell suspension and seeded onto a matrigel-coated plate in mTeSR1 medium supplemented with Rock inhibitor (Y-27632, final concentration 10 μM) to reach approximately 20% confluence on the following day. Mesoendoderm differentiation was initiated by treating hPSCs with RPMI/B-27 (RPMI/1640, 2% B-27 (minus insulin), 0.5% GlutaMax and 0.5% non-essential amino acid) supplemented with 10 μM CHIR99021 for one day. Cells were then treated with RPMI/B-27 supplemented with 20 ng/ml BMP4 for three more days to promote mesoderm generation. Alternatively, mesoderm cells were derived by treated cells with Mesoderm Induction medium. To induce differentiation of mesodermal progenitors, cells were grown in the RPMI/B-27 supplemented with 50 ug/ml of ascorbic acid, 0.5% ITS (insulin transferrin selenium), 0.5 μM dexamethasone, 5 ng/ml BMP4 and 20 ng/ml FGF1 for three days. Quiescent HSCs were finally induced by culturing cells in RPMI/B-27 supplemented with 50 ug/ml of ascorbic acid, 0.5% ITS, 0.5 μM dexamethasone, 1% synthetic lipids, 30 ng/ml EGF, 10 ng/ml FGF2, and 5 μM retinol for six days. A diagram of a simple differentiation protocol is included in FIG. 1A.

Differentiation of hPSC-Derived Hepatocyte (HEP)

Differentiation of HEP was performed as described previously [36]. Briefly, hPSCs were first differentiated into definitive endoderm cells by using the STEMdiff Definitive Endoderm Kit following the manufacturer's instructions. Then, endoderm cells were dissociated into single-cell suspensions using Accutase and plated onto Matrigel-coated plates with Rock inhibitor in RPMI/B-27 supplemented with 20 ng/ml BMP4 and 10 ng/ml FGF2. Medium was replaced daily for five days. Cells were then exposed to RPMI/B-27 containing 20 ng/ml HGF for five days. Cells were further matured in Lonza hepatocyte culture medium containing ascorbic acid, BSA-FAF, hydrocortisone, transferrin, insulin, GA-1000, and 20 ng/ml OSM for an additional one to two weeks. HEP cultures were replenished with fresh medium every two days.

Differentiation of hPSC-Derived Macrophages (Macs)

hPSCs were first differentiated into hematopoietic stem cells using the Spin-EB method. Briefly, 3000 hPSC cells were seeded into U-bottom 96-well non-tissue culture plates in 50 μL of SFM (IMDM/Ham's F12 (1:1), 5 mg/ml BSA, 1× insulin-transferrin-selenium, 1× synthetic lipids, 50 μg/ml of ascorbic acid and 2 mM GlutaMax) supplemented with 10 ng/ml BMP4, 10 ng/ml FGF2, and 10 μM Y-27632 for two days. SFM supplemented with 10 ng/ml BMP4, 10 ng/ml FGF2, and 20 ng/ml VEGF was added to cultures (50 μl/well) every three days. On day 8, half of the culture medium was removed and fresh SFM medium containing 10 ng/ml FGF2, 10 ng/ml VEGF, and 50 ng/ml SCF were added every three days until day 14 when cells were collected for purification of HSCs using the EasySep human CD34 positive selection kit (Stemcell Technologies) according to the manufacturer's instructions.

Purified hematopoietic stem cells were expanded in the expansion medium (StemSpan SFEM II supplemented with 10% CD34+ expansion supplement) for three days. Macrophage differentiation was initiated by culturing hematopoietic stem cells in induction medium (IMDM, 3% AB serum, 2% human plasma, 10 ng/ml insulin, 3U/ml heparin and 200 μg/ml transferrin) supplemented with 50 ng/ml SCF, 1 ng/ml IL-3, 50 ng/ml Flt3 and 100 ng/ml M-CSF for 7-11 days. Medium was replenished every three days and cultures were further expanded to keep the cell density around 1.0-1.5 million/ml.

### Establishment of a Multicellular Co-Culture

hPSC-derived HEPs, HSCs, and Mac were differentiated separately. HEPs from day 15 and HSCs from day 12 (or day 11 if using Mesoderm Induction medium) of differentiation were incubated with Accutase supplemented with 10 μM Y-27632 and detached into single-cell suspension. HEPs and HSCs were mixed at a ratio of 8:1 and re-plated onto matrigel coated 12-well cell culture plate, at a density of 0.4×10⁶ cells/well, in the medium (50% of the final stage medium for HEPs and 50% of the final stage medium for HSCs) supplemented with 10 μM Y-27632. The following day, Macs from day 9 of differentiation were added in a 1:1 ratio with HSCs to the top compartment of a transwell system and the transwell insert was placed onto the well containing HEPs/HSCs. Culture medium was changed to basal maintenance medium (BMM) that consisted of glucose-free DMEM supplemented with 2% knockout serum replacement (KOSR), 2% B-27, 3U/ml heparin, 200 μg/ml Transferrin, 30 ng/ml EGF, 5 μM retinol, and 0.5 μM dexamethasone. The 12-well plate containing co-cultures was placed on an orbital shaker platform at a speed of 30 rpm/min. Co-culture was replenished with fresh medium every two days or as indicated in the figure legend. To mimic healthy condition, BMM was supplemented with normal physiological levels of insulin (0.7 nM) and glucose (6.0 mM). To mimic lipotoxic condition, high levels of insulin (7.0 nM), glucose (25.0 mM) and free fatty acid (oleic acid, 68 μM and palmitic acid, 45 μM) to mimic plasma concentrations of these factors in NAFLD patients. In these experiments, half of the culture medium was replenished every two days.

Separation of HEPs from HSCs

Isolation of HEPs from co-cultures was performed as described previously with modifications [37]. Briefly, hPSC-derived co-cultures of HEPs and HSCs were washed once with Versene and incubated in Versene at 37° ° C. for 20-25 minutes to loosen the cell-to-cell contacts. Cells were washed with pre-warmed DMEM/F12 and then incubated with pre-warmed collagenase mixture (2.0 mg/ml collagenase, 1.0 mg/ml dispase, 100UI/ml DNase, 0.2% DMSO in HBM) at 37ºC for 35-40 minutes. During incubation, cells were gently pipetted to break up clumps and aid dissociation. Cells were then collected by adding Versene and centrifuged at 400 g for 5 minutes at room temperature. After resuspension in Versene, cells were further incubated at 37° C. for 45 minutes. Towards the end of Versene incubation, the majority of cells should be dissociated to single cells. If necessary, the single-cell suspension was further filtered using a 100 μm cell strainer to remove large cell clumps. To separate HEPs from HSCs, single cells were first blocked in 1% BSA and incubated on ice for 1.0 hr. Cells were then stained with mouse anti-ASGR1 antibody, incubated with anti-mouse IgG microbeads, and separated by a magnetic separator at 4° C. HEPs were collected from the fraction bound to the magnetic wall; HSCs were collected from the flow-through faction after another round of hepatocyte purification. Purified HEPs and HSCs were collected for downstream analysis including various metabolic assays, western blot, RNA extraction, and flow cytometry.

### In Vitro Activation of Quiescent HSCs

For replating-induced activation, hPSC-derived HSCs at day 13 of differentiation were detached by incubation with Accutase and re-plated onto cell culture-treated plastic surface in the basic medium (RPMI/B-27, 0.5% ITS, 1% synthetic lipids, and 0.5% GlutaMax). For TGFβ1, FBS-, PDGF- and LPS-induced activation, hPSC-derived HSCs were incubated with the abovementioned basic medium in the presence of 5 ng/ml TGFβ1, 10% FBS, 30 ng/ml PDGF, or 500 ng/ml LPS, and analyzed for expression of activation marker genes by RT-qPCR at 24 hrs or by western blot at the indicated time points post-treatment.

### Quantification of HSC Activation

To quantify the secretion of activation markers in the supernatant, activated HSCs were first washed with DMEM/F-12 three times before being incubated with medium (RPMI/1640 and 0.5% GlutaMax) for 3 hrs. The supernatants were analyzed by western blot using an anti-COL1A1 antibody. To monitor fibronectin (FN) secretion, the supernatant from multicellular cultures was collected and subject to analysis by western blot using an anti-FN antibody. To quantify the expression of intracellular activation markers, purified HSCs were directed lysed in 2× western blot lysis buffer and subject to analysis using specific antibodies.

### Triacylglycerol (TAG) Assay

Intracellular triacylglycerol levels were analyzed for hPSC-derived HEPs purified from different conditions using commercial kits (Promega) per the manufacturer's instructions.

### Glucose Secretion by HEPs

For measurement of basal levels of total glucose secretion by HEPs, the transwell insert with Macs was removed, HEPs and HSCs co-cultures were washed gently but thoroughly using glucose-free DMEM and incubated at 37° ° C. for 6 hrs in glucose-free DMEM. Supernatants were then collected and concentrated by nitrogen gas flow after which glucose levels were measured using a Glucose Detection Kit (Sigma) according to the manufacturer's instructions. For insulin-stimulated glucose secretion, HEPs and HSC co-cultures were washed as above, incubated with glucose-free DMEM supplemented with high glucose (25.0 mM) for 1 hr, and then incubated at 37° C. for 6 hrs in glucose-free DMEM in the presence or absence of insulin (200.0 nM). Supernatants were collected and glucose levels were measured as above.

### Measurement of CYP P450 Enzyme Activities in HEPs

CYP P450 enzymatic activities were analyzed for hPSC-derived HEPs at day 20 using commercial kits (Promega) per the manufacturer's instructions. Primary human hepatocytes from two different donors were included as a positive control.

### Measurement of Intracellular ATP in HEPs

Purified hPSC-derived HEPs and PHHs were washed with D-PBS twice and seeded into 96-well plate (3×10⁴/well). The intracellular ATP was measured using the CellTiter-Glo luminescent cell viability assay kit (Promega) according to the manufacturer's instructions.

### Phagocytosis Assay in Macs and Primary Kupffer Cells

Phagocytosis of green E. coli by hPSC-derived Macs was performed using a phagocytosis assay kit (BioVision) according to the manufacturer's instructions. Cytochalasin D was used as a phagocytosis inhibitor. Phagocytosis of green E. coli was analyzed using a fluorescent microscope.

### CRISPR Cas9-Mediated Gene Editing

We first generated hPSCs with doxycycline-inducible expression of Cas9 (HUES8-iCas9) using TALEN-mediated gene targeting into the AAVS1 locus. Then the iCas9 cells were genotyped by using PCR primers: sense: 5′-GACCTGCTGCATGGGAATTCTGGGG-3′ (SEQ ID NO:1) and anti-sense: 5′-CCAGCCAGTTTACCTTACA-3′ (SEQ ID NO:2) to amply a 722 bp fragment followed by sanger sequencing. The introduction of rs738409 C>G mutation was achieved as follow: Briefly, a single guide RNA (sgRNA) (sequence: TGCTTCATGCCTTTCTACAG (SEQ ID NO:3)) was cloned into pLenti-CRISPR-V2 vector and used as PCR template to amply a 120 bp fragment including the T7 promoter sequence, CRISPR recognition sequence, and a constant chimeric sgRNA sequence, using primers (sense: 5′-TAATACGACTCACTATAGGGACTGTAGAAAGG-3′ (SEQ ID NO:4) and antisense: 5′-AAAAGCACCGACTCGGTGCC-3′ (SEQ ID NO:5)). Purified PCR product was then used for in vitro transcription to generate sgRNA. For homology-directed repair, a single-stranded DNA (ssDNA) template (sequence: 5′-GAGCACACTTCAGAGGCCCCC AGGACTCAGCGCTAGCAGAGAAAGCCGACTTACCACGCCTCTGAAGGAAGGAGG GATAAGGCCACTGTAGAAAGGGATGAAGCACGAACATACCAAGGCCTGTGAAA GCAAAGGAGAGAGAAGTTATAGGCGAGAGCACCCTTTTAATTTTCCTGATCCTTC ATAAGCTTTC-3; (SEQ ID NO:6)) containing the mutated sequence flanked by 90nt of homology on each side was synthesized. Before ssDNA/sgRNA co-transfection, iCas9-hPSCs were pretreated with doxycycline (2 μg/ml) for 24 hrs. Cells were transfected with ssDNA/sgRNA (ratio 5:1) twice using RNAiMAX reagent (Invitrogen) in the presence of doxycycline (2 μg/ml). The first transfection was done while cells were being seeded. Two days after the second transfection, cells were detached into single-cell suspension and used for single-cell cloning. Clones were then picked, expanded, and analyzed by PCR using the two genotyping primers. Correct clones were further characterized for their growth rate, pluripotent marker expression, and trilineage differentiation capacity.

Trilineage Differentiation of hPSCs

Trilineage differentiation was done on the isogenic pair of hPSC using STEMdiff™ Trilineage Differentiation kit (Stemcell Technologies) per the manufacturer's instructions.

### Transcriptome Analyses (RNA-Seq)

Approximately 100 ng of total RNA was used as input. Libraries were prepared according to Illumina's instructions for the TruSeq Stranded mRNA LT Sample Prep Kit (Catalog IDs: RS-122-2101, RS-122-2102) and sequenced on an Illumina NextSeq 500 with a read length of 75 nucleotides (single end configuration).

Libraries were sequenced to generate approximately 20 to 30 million reads per sample. FastQC (v.0.11.8) was used to assess the quality of reads. Trimmomatic (v0.39) was used to trim the reads, and quality was assessed again with FastQC. Gene and transcript expression levels (TPM and estimated counts) were quantified by pseudo-alignment using Salmon (v0.13.1) using the annotation from Ensembl v95 as the reference transcriptome. The R statistical framework (v3.5.1) was used for all downstream analyses. The tximport package (v1.8.0) was used to import and summarize these estimates. For PCA analysis, the DESeq2 package (v1.20.0) was used to normalize and variance stabilize the estimated read counts by regularized log transformation prior to plotting using the ggplot2 package (v3.0.0). For heatmap analyses, data was plotted using the ggplot2 package (v3.1.1).

### Transcriptomic Data Analysis of Liver Biopsy Samples

Raw sequences for patient liver biopsy RNA-seq data were downloaded from GEO database (GSE126848 and GSE135251). The sequences were reprocessed through the nf-core/rnaseq pipeline [38]. The pipeline involved quality control of the reads with FastQC, adapter trimming using Trim Galore! (https://github.com/FelixKrueger/TrimGalore), read alignment with STAR [39]. Alignment was performed using the GRCh38 build native to nf-core and annotation was performed using Gencode Human Release 33 (GRCH38.p13). The quantification of the reads was done with FeatureCounts. The reads were normalized using variance-stabilizing transform (vst) in DESeq2 package in R for visualization purposes in log-scale [40]. Differential expression of genes was calculated by DESeq2.

The comparisons were done either by disease conditions or by SNP conditions (GSE135251 dataset only), correcting for sequencing batches with a covariate where applicable. To retrieve the SNP status of the PNPLA3 locus (chromosome 22, rs738409), the variant was called from the aligned sequences detailed above, using SAMtools mpileup and bcftools [41, 42]. The called variant was categorized as homozygous, heterozygous, and wild-type and stored as a metadata for downstream analyses.

Differentially expressed genes were determined by the cutoffs of BH-adjusted p-value <0.05 and absolute log 2 fold-change greater than 1. Among these differentially expressed genes, the top 100 variable genes were used to perform gene ontology (GO) analysis using enrichR [43]. Any signature with p-value <0.05 was taken as significant. A list of significantly enriched pathways is reported in Table 2.

### Immunofluorescent Analysis

Cells were fixed in 4% para-formaldehyde in phosphate-buffered saline (PBS) at room temperature for 10 min and blocked with PBTG (PBS containing 10% normal goat serum, 1% bovine serum albumin (BSA), 0.1% Triton-X100) at room temperature for 2 to 3 hr. Cells were incubated with primary antibodies at 4° C. overnight or 2 hr at room temperature. Isotype mouse or rabbit IgGs were used as negative controls. After four washes with PBS, Alexa-350, 488, or 594 conjugated secondary antibodies were added and incubated in the dark at room temperature for 1 hr, followed by four washes with PBS. Staining of hPSC-derived Macs was similarly performed, except cells were forced to attach to slides via cytospining. For lipid droplets staining in hPSC-derived HEPs, cells grown on coverslips were fixed with 10% formalin for 30 min and washed with 60% isopropanol for 10 min at room temperature. Cells were then stained with 0.18% freshly made oil-red in PBS for 5 min at room temperature and washed thoroughly with double-distilled water four times. Nuclei were stained with DAPI for 1 min at room temperature. Oil red quantification was performed as total red staining intensity normalized to nuclei counts using ImageJ software. Representative images from at least three independent experiments are shown in figures.

### Flow Cytometry

Expression of surface markers on hPSC-derived Macs was assessed by flow cytometry. Specifically, Macs were first purified from hPSC-derived macrophage cultures using anti-CD163 antibody-conjugated magnetic beads. Purified cells were then stained with fluorescent dye-conjugated anti-CD14, anti-CD11b, anti-CD68, and anti-CD163 antibodies at 4° C. in dark. Dead cells were excluded during flow cytometry analysis and gating was determined by using isotype controls.

To quantify retinol content, HSCs in single-cell suspension were analyzed for auto-fluorescence after UV light excitation using FACS-LSRII (BD Bioscience) and mean fluorescent intensity was calculated. For gating under UV light, mesoderm (MES) cells were used as a negative control. Stained cells were analyzed with FACS-LSRII (BD Bioscience).

### Quantitative Real-Time RT-PCR (RT-qPCR)

Total RNA was isolated from cell lysates using the RNAeasy Mini Kit (Qiagen) followed by reverse transcription using Superscript III First-Strand Synthesis System or RevertAid First Strand cDNA Synthesis (ThermoFisher). Gene expression was quantified using the LightCycler SYBR Green I Master Mix (Roche Life Science) on a LightCycler 480 Instrument (Roche Life Science) or QuantStudio3 Instrument (Applied Biosystems) with gene-specific primers. PCR conditions were as follows: initial denaturation step at 50° C. for 2 min and 95° C. for 10 min, then 45 cycles of 95° C. for 15 sec, 56° C. for 15 sec, and 72° C. for 20 sec; followed by a melting step of 95° C. for 10s, 65° C. for 10s and a 0.07° C./s decrease from 95° C.; and finally, a cooling step of 50° C. for 5s. PCR product specificity was confirmed by a melting-curve analysis. Fold changes in mRNA expression were determined using the ΔΔCt method relative to the values in control samples as indicated in figure legends, after normalization to housekeeping genes (RPS11 or GAPDH). Results are presented as means±standard deviation (SD), unless stated otherwise. Comparisons between groups/cells were made using the two-tailed t-test with Welch's corrections to calculate exact p-values, unless stated otherwise. Statistical analysis was performed in Graph Pad PRISM 8.4.

### Western Blot Analysis

Cells were directly lysed in 2×SDS lysis buffer and cell lysates were separated by 7.5%, 10%, or 12% sodium dodecyl sulfate-polyacrylamide gel electrophoresis, in MES or MOPS buffer, followed by transfer onto polyvinylidene fluoride membrane (EMD Millipore), as previously described. To detect phosphorylated AKT and phosphorylated FOXO1 in HEPs, cells were washed with glucose-free DMEM, and incubated at 37° C. for 2 hrs in glucose-free DMEM supplemented with glucose (6.0 mM), then either left untreated or treated with 10.0 nM insulin for 10 minutes at 37° C. followed by being lysed in 2×SDS sample buffer (2 mL Tris-HCl (pH 6.8), 50% glycerol, 10% SDS, 0.5% bromophenol blue, and freshly added 10% β-mercaptoethanol). Representative blots from at least three independent experiments are shown in figures.

For comparison between different groups or treatments, β-actin (ACTB) or RPS11 were used as housekeeping gene controls.

### Viruses

The Sendai virus-Cantell stock was propagated by inoculation into the allantoic cavity of 10-day-old embryonated chicken eggs. Following incubation at 37° ° C. for 48 h, allantoic fluid was harvested and titered by hemagglutination of chicken red blood cells. The influenza A/PR/8/34-ΔNS1 virus (kindly provided by Adolfo Garcia-Sastre) was grown in MDCK cells constitutively expressing the influenza virus NS1 protein (MDCK-NS1). Cells were infected at a multiplicity of infection (MOI) of 0.01 and the supernatant was harvested 5 days post-infection when the cytopathic effect was detected. The stock titer was determined by titrating the virus on MDCK-NS1 cells followed by immunostaining for viral NP. Both viral stocks were aliquoted and stored at −80° C.

hPSC-derived and primary macrophages and HSCs were stimulated with either Sendai or influenza particles for 12 hrs and analyzed for transcript levels of various cytokine genes by RT-qPCR.

### TGFB1, IL6, and IL1β ELISA

To measure the secreted levels of human cytokines TGFβ1, IL6, and IL1β in the liver cultures under different experiment settings, supernatants were collected at indicated time points and cytokines levels were quantified by ELISA kits (R&D Systems), according to the manufacturers' instructions.

### Cell Migration Assay

hPSC-derived and primary HSCs were cultured in RPMI/B-27 (RPMI/1640, 2% B-27 (minus insulin), 0.5% GlutaMax and 0.5% non-essential amino acid) on plastic surface. Once the cells reached confluence, the monolayer was scraped with a 200-pipet tip to create a scratch. Following removal of debris, cells were cultured in fresh medium supplemented with PDGF-BB (25 ng/ml), FBS (10%), or Y-27532 (20 μM) for 48 hrs. Using phase contrast microscopy, images were taken at 0 hr (after debris removal) and 48 hrs after scratching. Closure distances were calculated as percentage with respect to time=0 distance.

IL6 Neutralization Assay and sgp130Fc Treatment

For IL6 neutralization and sgp130-Fc treatment in the multicellular liver cultures, IL6 neutralizing antibody and sgp130Fc protein were added directly to culture supernatant to a final concentration of 100 ng/ml (IL6 neutralization) or 10-100 ng/ml (spg130Fc) and replenished whenever a new culture medium was added.

Soluble IL6R (sIL6R) and Soluble Gp130 (Sgp130) Measurement

The supernatants were collected at indicated experiment settings in liver culture, and sIL6R and sgp130 levels were quantified by ELISA kits (Invitrogen), according to the manufacturers' instructions.

### Periodic Acid-Schiff (PAS) and Indocyanine Green (ICG) Staining

PAS staining was done on hPSC-derived HEPs at day 20 using a commercial kit (Sigma-Aldrich) per the manufacturer's instructions. ICG was first dissolved in DMSO and then further by culture medium to a final concentration of 1 mg/ml. HEPs were incubated with ICG for 30 min at 37° C. Then the uptake of ICG by HEPs was observed using a microscope following washing cells with culture medium twice. Cells were then maintained in a 37° C. incubator for another 12 hrs for the second check of ICG.

NF-κB reporter assay

The fragment containing five copies of NF-κB binding sites (GGGGACTTTCCGC) with firefly luciferase was amplified from psiCHECK2-NF-κB (from Q. Deng at Purdue University) by PCR and cloned into pLenti-CRISPR v2 vector to construct pLenti-NF-κB-FLuc. Macrophages derived from hPSC^{WT} and hPSC^{I148M} were transduced with pLenti-NF-κB-FLuc before being incorporated into liver cultures. Macrophages were then collected for luciferase reporter assay at two weeks post lipotoxic condition exposure.

### Cell Death Measurements

To measure hepatocyte cell death under lipotoxic condition, the supernatants and cells were collected at indicated experiment conditions, then lactate dehydrogenase (LDH) activity and caspase-generate CK18 fragments were quantified by LDH cytotoxicity assay kit (Invitrogen) and M30 CytoDeath CK18 kit (Diapharma), according to the manufacturers' instructions.

### Statistical Analysis

The sample sizes were determined by power analysis based on pilot data collected in our laboratory or published studies (PMC3320597, PMC5811326, PMC7125181). The exact value of n, and what n represents (e.g., number of cell clones or experimental replicate) can be found in figure legends. The data were analyzed using GraphPad Prism 8.4.3. software. The unpaired Student's t test (two-tailed) was used for comparisons between two groups.

Comparisons between three or more independent groups were performed using one-way analysis of variance (ANOVA) with Tukey's multiple comparison's test, unless stated otherwise. The data are presented as the mean±Standard deviation (SD) in graphs. All p-values were shown and values of p<0.05 were considered statistically significant.

### REFERENCES

[1] Loomba R, Friedman S L, Shulman G I. Mechanisms and disease consequences of nonalcoholic fatty liver disease. Cell 2021; 184:2537-2564.
[2] Trépo E, Valenti L. Update on NAFLD genetics: From new variants to the clinic. Journal of Hepatology 2020; 72:1196-1209.
[3] Friedman S L, Neuschwander-Tetri B A, Rinella M, Sanyal A J. Mechanisms of NAFLD development and therapeutic strategies. Nature Medicine 2018; 24:908-922.
[4] Romeo S, Kozlitina J, Xing C, Pertsemlidis A, Cox D, Pennacchio L A, et al. Genetic variation in PNPLA3 confers susceptibility to nonalcoholic fatty liver disease. Nature Genetics 2008; 40:1461-1465.
[5] Abul-Husn N S, Cheng X, Li A H, Xin Y, Schurmann C, Stevis P, et al. A Protein-Truncating HSD17B13 Variant and Protection from Chronic Liver Disease. N Engl J Med 2018; 378:1096-1106.
[6] Chitturi S, Abeygunasekera S, Farrell G C, Holmes-Walker J, Hui J M, Fung C, et al. NASH and insulin resistance: Insulin hypersecretion and specific association with the insulin resistance syndrome. Hepatology 2002; 35:373-379.
[7] Tavares De Almeida I, Cortez-Pinto H, Fidalgo G, Rodrigues D, Camilo M E. Plasma total and free fatty acids composition in human non-alcoholic steatohepatitis. Clinical Nutrition 2002; 21:219-223.
[8] Sookoian S, Pirola C J. Meta-analysis of the influence of I148M variant of patatin-like phospholipase domain containing 3 gene (PNPLA3) on the susceptibility and histological severity of nonalcoholic fatty liver disease. Hepatology 2011; 53:1883-1894.
[9] Govaere O, Cockell S, Tiniakos D, Queen R, Younes R, Vacca M, et al. Transcriptomic profiling across the nonalcoholic fatty liver disease spectrum reveals gene signatures for steatohepatitis and fibrosis. Science Translational Medicine 2020; 12:eaba4448.
[10] Tilson S G, Morell C M, Lenaerts A-S, Park S B, Hu Z, Jenkins B, et al. Modeling PNPLA3-Associated NAFLD Using Human-Induced Pluripotent Stem Cells. Hepatology 2021.
[11] Arab J P, Arrese M, Trauner M. Recent Insights into the Pathogenesis of Nonalcoholic Fatty Liver Disease. Annu Rev Pathol 2018; 13:321-350.
[12] Coll M, Perea L, Boon R, Leite S B, Vallverdú J, Mannaerts I, et al. Generation of Hepatic Stellate Cells from Human Pluripotent Stem Cells Enables In Vitro Modeling of Liver Fibrosis. Cell Stem Cell 2018; 23:101-113.e107.
[13] El Taghdouini A, Najimi M, Sancho-Bru P, Sokal E, van Grunsven L A. In vitro reversion of activated primary human hepatic stellate cells. Fibrogenesis & Tissue Repair 2015; 8:14.
[14] Si-Tayeb K, Lemaigre F P, Duncan S A. Organogenesis and Development of the Liver. Developmental Cell 2010; 18:175-189.
[15] Bilzer M, Roggel F, Gerbes A L. Role of Kupffer cells in host defense and liver disease. Liver Int 2006; 26:1175-1186.
[16] Sharifnia T, Antoun J, Verriere T G C, Suarez G, Wattacheril J, Wilson K T, et al. Hepatic TLR4 signaling in obese NAFLD. Am J Physiol Gastrointest Liver Physiol 2015; 309:G270-G278.
[17] Lin Hua V, Accili D. Hormonal Regulation of Hepatic Glucose Production in Health and Disease. Cell Metabolism 2011; 14:9-19.
[18] Feaver R E, Cole B K, Lawson M J, Hoang S A, Marukian S, Blackman B R, et al. Development of an in vitro human liver system for interrogating nonalcoholic steatohepatitis. JCI Insight 2016; 1.
[19] Johansen J S, Christoffersen P, Møller S, Price P A, Henriksen J H, Garbarsch C, et al. Serum YKL-40 is increased in patients with hepatic fibrosis. Journal of Hepatology 2000; 32:911-920.
[20] Tanaka T, Narazaki M, Kishimoto T. IL-6 in Inflammation, Immunity, and Disease. Cold Spring Harbor Perspectives in Biology 2014; 6.
[20] Wieckowska A, Papouchado B G, Li Z, Lopez R, Zein N N, Feldstein A E. Increased Hepatic and Circulating Interleukin-6 Levels in Human Nonalcoholic Steatohepatitis. Am J Gastroenterol 2008; 103:1372-1379.
[22] Pirazzi C, Valenti L, Motta B M, Pingitore P, Hedfalk K, Mancina R M, et al. PNPLA3 has retinyl-palmitate lipase activity in human hepatic stellate cells. Human Molecular Genetics 2014; 23:4077-4085.
[23] BasuRay S, Smagris E, Cohen J C, Hobbs H H. The PNPLA3 variant associated with fatty liver disease (I148M) accumulates on lipid droplets by evading ubiquitylation. Hepatology 2017; 66:1111-1124.
[24] Suppli M P, Rigbolt K T G, Veidal S S, Heebøll S, Eriksen P L, Demant M, et al. Hepatic transcriptome signatures in patients with varying degrees of nonalcoholic fatty liver disease compared with healthy normal-weight individuals. Am J Physiol Gastrointest Liver Physiol 2019; 316:G462-G472.
[25] Luukkonen P K, Nick A, Hölttä-Vuori M, Thiele C, Isokuortti E, Lallukka-Brück S, et al. Human PNPLA3-I148M variant increases hepatic retention of polyunsaturated fatty acids. JCI Insight 2019; 4.
[26] Giraldez M D, Carneros D, Garbers C, Rose-John S, Bustos M. New insights into IL-6 family cytokines in metabolism, hepatology and gastroenterology. Nat Rev Gastroenterol Hepatol 2021; 18:787-803.
[27] Jones S A, Jenkins B J. Recent insights into targeting the IL-6 cytokine family in inflammatory diseases and cancer. Nat Rev Immunol 2018; 18:773-789.
[28] Garbers C, Heink S, Korn T, Rose-John S. Interleukin-6: designing specific therapeutics for a complex cytokine. Nat Rev Drug Discov 2018; 17:395-412.
[29] Collin de l'Hortet A, Takeishi K, Guzman-Lepe J, Morita K, Achreja A, Popovic B, et al. Generation of Human Fatty Livers Using Custom-Engineered Induced Pluripotent Stem Cells with Modifiable SIRT1 Metabolism. Cell Metabolism 2019; 30:385-401.e389.
[30] Matthews V B, Allen T L, Risis S, Chan M H S, Henstridge D C, Watson N, et al. Interleukin-6-deficient mice develop hepatic inflammation and systemic insulin resistance. Diabetologia 2010; 53:2431-2441.
[31] Schreiber S, Aden K, Bernardes J P, Conrad C, Tran F, Hoper H, et al. Therapeutic Interleukin-6 Trans-signaling Inhibition by Olamkicept (sgp130Fc) in Patients With Active Inflammatory Bowel Disease. Gastroenterology 2021; 160:2354-2366 e2311.
[32] Bergmann J, Muller M, Baumann N, Reichert M, Heneweer C, Bolik J, et al. IL-6 trans-signaling is essential for the development of hepatocellular carcinoma in mice. Hepatology 2017; 65:89-103.
[33] Bekkering S, Quintin J, Joosten L A, van der Meer J W, Netea M G, Riksen N P. Oxidized low-density lipoprotein induces long-term proinflammatory cytokine production and foam cell formation via epigenetic reprogramming of monocytes. Arterioscler Thromb Vasc Biol 2014; 34:1731-1738.
[34] Colak Y, Senates E, Ozturk O, Doganay H L, Coskunpinar E, Oltulu Y M, et al. Association of serum lipoprotein-associated phospholipase A2 level with nonalcoholic fatty liver disease. Metab Syndr Relat Disord 2012; 10:103-109.
[35] Triggiani M, Granata F, Oriente A, Gentile M, Petraroli A, Balestrieri B, et al. Secretory phospholipases A2 induce cytokine release from blood and synovial fluid monocytes. Eur J Immunol 2002; 32:67-76.
[36] Wu X, Dao Thi V L, Huang Y, Billerbeck E, Saha D, Hoffmann H-H, et al. Intrinsic Immunity Shapes Viral Resistance of Stem Cells. Cell 2018; 172:423-438.e425.
[37] Mallanna Sunil K, Cayo Max A, Twaroski K, Gundry Rebekah L, Duncan Stephen A. Mapping the Cell-Surface N-Glycoproteome of Human Hepatocytes Reveals Markers for Selecting a Homogeneous Population of iPSC-Derived Hepatocytes. Stem Cell Rep 2016; 7:543-556.
[38] Ewels P A, Peltzer A, Fillinger S, Patel H, Alneberg J, Wilm A, et al. The nf-core framework for community-curated bioinformatics pipelines. Nat Biotechnol 2020; 38:276-278.
[39] Dobin A, Davis C A, Schlesinger F, Drenkow J, Zaleski C, Jha S, et al. STAR: ultrafast universal RNA-seq aligner. Bioinformatics 2012; 29:15-21.
[40] Love M I, Huber W, Anders S. Moderated estimation of fold change and dispersion for RNA-seq data with DESeq2. Genome Biol 2014; 15:550.
[41] Li H, Handsaker B, Wysoker A, Fennell T, Ruan J, Homer N, et al. The Sequence Alignment/Map format and SAMtools. Bioinformatics 2009; 25:2078-2079.
[42] Danecek P, Bonfield J K, Liddle J, Marshall J, Ohan V, Pollard M O, et al. Twelve years of SAMtools and BCFtools. GigaScience 2021; 10.
[43] Chen E Y, Tan C M, Kou Y, Duan Q, Wang Z, Meirelles G V, et al. Enrichr: interactive and collaborative HTML5 gene list enrichment analysis tool. BMC Bioinformatics 2013; 14:128.

### SUPPLEMENTARY REFERENCES

[1] Wu X, Dao Thi V L, Huang Y, Billerbeck E, Saha D, Hoffmann H-H, et al. Intrinsic Immunity Shapes Viral Resistance of Stem Cells. Cell 2018; 172:423-438.e425.
[2] Mallanna Sunil K, Cayo Max A, Twaroski K, Gundry Rebekah L, Duncan Stephen A. Mapping the Cell-Surface N-Glycoproteome of Human Hepatocytes Reveals Markers for Selecting a Homogeneous Population of iPSC-Derived Hepatocytes. Stem Cell Rep 2016; 7:543-556.
[3] Ewels P A, Peltzer A, Fillinger S, Patel H, Alneberg J, Wilm A, et al. The nf-core framework for community-curated bioinformatics pipelines. Nat Biotechnol 2020; 38:276-278.
[4] Dobin A, Davis C A, Schlesinger F, Drenkow J, Zaleski C, Jha S, et al. STAR: ultrafast universal RNA-seq aligner. Bioinformatics 2012; 29:15-21.
[5] Love M I, Huber W, Anders S. Moderated estimation of fold change and dispersion for RNA-seq data with DESeq2. Genome Biol 2014; 15:550.
[6] Li H, Handsaker B, Wysoker A, Fennell T, Ruan J, Homer N, et al. The Sequence Alignment/Map format and SAMtools. Bioinformatics 2009; 25:2078-2079.
[7] Danecek P, Bonfield J K, Liddle J, Marshall J, Ohan V, Pollard M O, et al. Twelve years of SAMtools and BCFtools. GigaScience 2021; 10.
[8] Chen E Y, Tan C M, Kou Y, Duan Q, Wang Z, Meirelles G V, et al. Enrichr: interactive and collaborative HTML5 gene list enrichment analysis tool. BMC Bioinformatics 2013; 14:128.

All publications and patents mentioned in the specification and/or listed below are herein incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the relevant fields are intended to be within the scope described herein.

We claim:

1. A method of maintaining quiescence in quiescent hepatic stellate cells (HSCs) comprising:
culturing quiescent HSCs in culture media for a time period such that said quiescent HSCs remain quiescent for said time period,
wherein said time period is at least one day, and
wherein said culture media comprises at least one of the following: fibroblast growth factor 1 (FGF1), fibroblast growth factor 2 (FGF2), and retinol.

2. The method of claim 1, wherein said culture media comprises all three of FGF1, FGF2, and retinol.

3. The method of claim 1, wherein said culture media further comprises epidermal growth factor (EGF) and/or lipids, wherein said EGF is optionally present in said culture media at a concentration of 5-120 ng/ml or 25-35 ng/ml, and optionally, wherein said lipids comprise a mixture of at least three of the following: arachidonic acid, linoleic acid, linolenic acid, myristic acid, oleic acid, palmitic acid stearic acid, cholesterol, Tween-80, tocopherol acetate and Pluronic F-68.

4. The method of claim 1, wherein said time period is at least 2 days, or at least 7 days, or at least 14 days or at least 28 days.

5. The method of claim 1, wherein said culture media comprises said FGF1, and wherein said FGF1 is present in said culture media at a concentration of about 1-80 ng/ml or 3-25 ng/ml.

6. The method of claim 1, wherein said culture media comprises said FGF2 and wherein said FGF2 is present in said culture media at a concentration of about 2-40 ng/ml or 7-13 ng/ml.

7. The method of claim 1, wherein said culture media comprises said retinol and wherein said retinol is present in said culture media at a concentration of about 1-20 μM or about 3-7 μM.

8. A composition or kit comprising:
a) quiescent hepatic stellate cells (HSCs), and
b) culture medium comprising at least one of the following: fibroblast growth factor 1 (FGF1), fibroblast growth factor 2 (FGF2), and retinol.

9. The composition or kit of claim 8, wherein said culture media comprises all three of FGF1, FGF2, and retinol.

10. The composition or kit of claim 8, wherein said culture media further comprises epidermal growth factor (EGF) and/or lipids, wherein said EGF is optionally present in said culture media at a concentration of 5-120 ng/ml or 25-35 ng/ml, and optionally, wherein said lipids comprise a mixture of at least three of the following: arachidonic acid, linoleic acid, linolenic acid, myristic acid, oleic acid, palmitic acid stearic acid, cholesterol, Tween-80, tocopherol acetate and Pluronic F-68.

11. The composition or kit of claim 8, wherein said culture media comprises said FGF1, and wherein said FGF1 is present in said culture media at a concentration of about 1-80 ng/ml or 3-25 ng/ml.

12. The composition or kit of claim 8, wherein said culture media comprises said FGF2 and wherein said FGF2 is present in said culture media at a concentration of about 2-40 ng/ml or 7-13 ng/ml.

13. The composition or kit of claim 8, wherein said culture media comprises said retinol and wherein said retinol is present in said culture media at a concentration of about 1-20 μM or about 3-7 μM.

14. A method comprising:
a) adding a single-cell suspension of human pluripotent stem cells (hPSCs) to a cell culture container containing a first cell-culture media,
wherein said first cell-culture media optionally contains a p160ROCK inhibitor, wherein said p160ROCK inhibitor optionally comprises Y-27632;
b) incubating at least a portion of said hPSCs such that at least about 10% confluence of said hPSCs is achieved;
c) treating at least a portion of said hPSCs with:
i) a second cell-culture media comprising at least one of the following: biotin, vitamin B12, and PABA, wherein said second cell-culture media optionally comprising RPMI 1640,
ii) a first media supplement comprising at least 5, 6, 7, 8, 9, 10, 11, 12, 13, 14, 15, 16, 17, 18, 19, or 20 reagents from Table 1, and optionally wherein said first media supplement comprises B-27 media supplement without insulin,
iii) optionally a second media supplement comprising L-alanyl-L-glutamine dipeptide, and wherein optionally said second media supplement comprises GlutaMax,
iv) optionally a third media supplement comprising non-essential amino acids;
v) optionally an inhibitor of the GSK-3 enzyme, wherein said inhibitor optionally comprises CHIR99021;
d) treating at least a portion of said hPSCs with either: i) said second cell-culture media, said first media supplement, and BMP4, and/or ii) Mesoderm Induction Medium; and
e) incubating at least a portion of said hPSCs such that mesoderm cells are generated.

15. The method of claim 14, further comprising:
f) treating at least a portion of said mesoderm cells with: said second cell-culture media, said first media, BMP4, FGF1, insulin, transferrin, and selenite, and optionally ascorbic acid and/or dexamethasone; and
g) culturing at least a portion of said mesoderm cells such that mesodermal progenitor cells are generated.

16. The method of claim 15, further comprising:
h) treating at least a portion of said mesodermal progenitor cells with: said second cell-culture media, said first media, insulin, transferrin, selenite, lipids, and FGF2, and optionally ascorbic acid, dexamethasone, and/or EGF, and
i) culturing at least a portion of said mesodermal progenitor cells such that quiescent hepatic stellate cells (HSCs) are generated.

17. The method of claim 15, wherein said lipids are selected from one or more of the following: arachidonic acid, linoleic acid, linolenic acid, myristic acid, oleic acid, palmitic acid, stearic acid, cholesterol, Tween-80, tocopherol acetate, Pluronic F-68 solubilized in cell culture water.
